(19) United States
(12) Patent Application Publication
(10) Pub. No.: US 2024/0207196 A1
(43) Pub. Date: Jun. 27, 2024

(54) COMPOSITIONS OF GLYCOPROTEIN PARTICLES

(71) Applicant: Scribe Therapeutics Inc., Alameda, CA (US)

(72) Inventors: Benjamin OAKES, El Cerrito, CA (US); Gayathri VIJAYAKUMAR, Oakland, CA (US)

(21) Appl. No.: 18/568,029

(22) PCT Filed: Jun. 7, 2022
(86) PCT No.: PCT/US2022/032577

§ 371 Date: Dec. 7, 2023

Related U.S. Application Data
(60) Provisional application No. 63/208,930, filed on Jun. 9, 2021.

(51) Int. Cl. A61K 9/51; A61K 48/00; C12N 7/00; C12N 9/22; C12N 15/11

(52) U.S. Cl. CPC A61K 9/5184; A61K 48/0033; A61K 48/0041; C12N 7/00; C12N 9/22; C12N 15/11; C12N 2310/20; C12N 2760/20223; C12N 2800/80

(57) ABSTRACT

Provided herein are glycoprotein particles comprising one or more glycoproteins, or fragments thereof that enhance targeting of a particle to a cell or tissue. A glycoprotein particle as provided herein may be used for any application, including therapeutic and diagnostic treatments.

### CROSS REFERENCE TO RELATED APPLICATIONS

This application claims priority to U.S. Provisional Patent Application No. 62/208,930, filed Jun. 9, 2021, the contents of which are incorporated by reference in their entirety.

### INCORPORATION BY REFERENCE OF SEQUENCE LISTING

This application contains a Sequence Listing which has been submitted in ASCII format via EFS-WEB and is hereby incorporated by reference in its entirety. Said ASCII copy, created on Jun. 5, 2022 is named SCRB_037_01WO_SeqList_ST25.txt and is 5.67 MB in size.

### BACKGROUND

Targeted delivery of therapeutic payloads to particular cells or tissues of the body generally requires complex systems involving the incorporation of a targeting modality. Even with highly selective targeting modalities, such as monoclonal antibodies, the selectivity of the system for the target cells or tissues is not absolute, and non-specific delivery and off-target toxicity may result. Thus, there is a need for additional compositions and methods to target therapeutic payloads to particular cell and tissue types.

### SUMMARY

The present disclosure provides glycoprotein particles, i.e., particles comprising a viral glycoprotein, to selectively target cells and tissues. As provided herein, a glycoprotein particle comprises a particle and a glycoprotein, wherein the glycoprotein is any one of the sequences of SEQ ID NOS: 1-224, 281-284, or 286-573 as set forth in Table 1, a sequence comprising at least 70%, at least 80%, at least 90%, at least 95%, at least 96%, at least 97%, at least 98%, or at least 99% identity thereto, or a fragment thereof. In some embodiments, the glycoprotein particle comprises a glycoprotein consisting of any one of SEQ ID NOS: 1-224, 281-284, or 286-573 as set forth in Table 1. In some embodiments, the glycoprotein fragment is a virion surface domain.

In some embodiments, the glycoprotein particle comprises a microparticle, a nanoparticle, or one or more of fullerenes, metals, biological polymers, dendrimers, quantum dots, lipids, nucleic acid vectors, and viral structural proteins.

In some embodiments, the glycoprotein particle comprises a polymeric nanoparticle, e.g., a nanocapsule or a nanosphere. In some embodiments, the glycoprotein particle comprises a polymer micelle. In some embodiments, the glycoprotein particle comprises a particle that is non-toxic and/or biodegradable. In some embodiments, the glycoprotein particle comprises a lipid-based particle.

In some embodiments, the glycoprotein particle comprises a virus-like particle (VLP). In some embodiments, the glycoprotein particle comprises a VLP derived from a virus selected from the group consisting of retrovirus, adeno-associated virus (AAV), adenovirus, and herpes simplex virus. In some embodiments, the retrovirus is an Orthoretrovirinae virus or a Spumaretrovirinae virus. In some embodiments, the Orthoretrovirinae virus is selected from the group consisting of an Alpharetrovirus, Betaretrovirus, Deltaretrovirus, Epsilonretrovirus, Gammaretrovirus, and Lentivirus. In some embodiments, the Spumaretrovirinae virus is selected from the group consisting of Bovispumavirus, Equispumavirus, Felispumavirus, Prosimiispumavirus, Simiispumavirus, or Spumavirus.

In some embodiments, the glycoprotein particle comprises an exosome. In some embodiments, the glycoprotein is expressed in a host cell and is trafficked to the host cell membrane prior to formation of the glycoprotein particle. In some embodiments, the glycoprotein particle is a lipid nanoparticle (LNP). In some embodiments, the glycoprotein is incorporated into a membrane of the glycoprotein particle.

In some embodiments, the glycoprotein particle comprises an organic polymer-based particle. In some embodiments, the glycoprotein particle comprises an inorganic nanoparticle. In some embodiments, the glycoprotein is conjugated to the surface of the glycoprotein particle. In some embodiments, the glycoprotein particle conjugation comprises covalent attachment or non-covalent attachment.

In some embodiments, glycoprotein particle comprises a payload. In some embodiments, inclusion of the glycoprotein in the glycoprotein particles enhances delivery of the payload to a cell or tissue compared to an equivalent particle that does not comprise the glycoprotein. In some embodiments, the payload comprises a protein, nucleic acid, small molecule, or combinations thereof. In some embodiments, the payload is a therapeutic payload. In some embodiments, delivery of the therapeutic payload to a cell or a tissue of a subject treats a disease or disorder in the subject.

In some embodiments, the therapeutic payload comprises a protein or an oligonucleotide. In some embodiments, the oligonucleotide comprises a sequence encoding a protein. In some embodiments, the protein comprises a cytokine, growth factor, interleukin, enzyme, receptor, microprotein, hormone, RNAse, DNAse, blood clotting factor, anticoagulant, bone morphogenetic protein, engineered protein scaffold, thrombolytics, antibody, antibody fragment, antibody fusion protein, transcription factor, viral interferon antagonist, tick protein, or engineered therapeutic protein.

In some embodiments, the oligonucleotide comprises a single-stranded antisense oligonucleotide (ASO), double-stranded RNA interference (RNAi) molecule, DNA aptamer, RNA aptamer, microRNA, ribozyme, RNA decoy, or circular RNA.

In some embodiments, the payload is a diagnostic payload.

In some embodiments, the payload comprises a gene editing system. In some embodiments. the gene editing system comprises a Class 2, Type II CRISPR/Cas system, a Class 2, Type V CRISPR/Cas system, a zinc finger nuclease or a TALEN. In some embodiments, the Class 2, Type II CRISPR/Cas system comprises Cas9. In some embodiments, the Class 2, Type V CRISPR/Cas system comprises one or more of CasX, Cas12a (Cpf1), Cas12b (C2c1), Cas12c (C2c3), Cas12d (CasY), Cas12e (CasX), Cas12f, Cas12g, Cas12h, Cas12i, Cas12j, Cas12k, Cas14, and/or CasΦ.

In some embodiments, the Class 2, Type V CRISPR/Cas system comprises a gene editing pair comprising a CasX protein comprising an amino acid sequence with at least 90% identity to any one of SEQ ID NOS: 574-944, or as set forth in Table 2, and a guide RNA (gRNA) comprising a scaffold comprising a nucleic acid sequence with at least 90% identity to any one of SEQ ID NOS: 945-1141, or as set forth in Table 3. In some embodiments, the payload comprises a gene editing pair comprising a CasX protein of any one of SEQ ID NOS: 574-944, or as set forth in Table 2, and a guide RNA comprising a scaffold of any one of SEQ ID NOS: 945-1141, or as set forth in Table 3. In some embodiments, the gRNA further comprises a targeting sequence linked to the 3′ end of the gRNA scaffold sequence.

In some embodiments, provided herein is a pharmaceutical composition comprising a glycoprotein particle and a pharmaceutically acceptable carrier, diluent, or excipient.

In some embodiments, provided herein is a method of treating a subject in need thereof, comprising administering to a subject in need thereof, a glycoprotein particle or a pharmaceutical composition thereof. In some embodiments, the subject has a disease or disorder selected from the group consisting of cancer, an immunoregulatory disease, a pulmonary disease or disorder, a cardiovascular disease, an infectious disease, a genetic disease or disorder, a neurological disease or disorder, an endocrine disease or disorder, a metabolic disease or disorder, an intestinal disease or disorder, a mental illness, a sexually transmitted disease, a gynecological disease, an urogenital disease, a skin disease, or an ocular disease. In some embodiments, administration of the glycoprotein particle or pharmaceutical composition reduces a sign or a symptom of the disease or disorder.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the results of percentage editing in mouse tdTomato neural progenitor cells (NPCs) with glycoprotein particles comprising viral-like particles (VLPs) comprising serial concentrations of the vesicular stomatitis virus glycoprotein (VSV-G), and a bald negative control VLP, wherein bald refers to a VLP lacking a glycoprotein, as described in Example 1.

FIG. 2 shows the results of size distributions and viral titer comparisons of glycoprotein particles comprising lentivirus VLPs comprising VSV-G and rabies glycoproteins, and a bald negative control lentivirus, as described in Example 1.

FIG. 3 depicts plasmids utilized in the creation of example glycoprotein particles comprising VLPs, with protease cleavage sites indicated by arrows, as described in Example 2.

FIG. 4 is a graph of editing results of example glycoprotein particles comprising VLPs having various incorporated glycoproteins, and a bald negative control, where bald refers to a VLP) lacking a glycoprotein, used to edit tdTomato in NPCs as measured by the number of particles (titer) added to treat the cells, as described in Example 2.

FIG. 5 is a graph of editing results with glycoprotein particles comprising VLPs having various incorporated glycoproteins, and a bald negative control VLP, used to edit tdTomato in NPCs as measured by the volume of VLPs added to treat the cells (μl of virus, x-axis), as described in Example 2.

FIG. 6 is a graph of editing results with glycoprotein particles comprising VLPs having various incorporated glycoproteins, and a bald negative control VLP, used to edit tdTomato in NPCs using 2 different volumes of VLPs to treat the cells, as described in Example 2. Glycoprotein constructs are provided in Table 4.

FIG. 7 is a scatterplot of EC₅₀ values for editing results with glycoprotein particles comprising VLPs having various incorporated glycoproteins, and a bald negative control, used to edit tdTomato in NPCs, as described in Example 2. The EC₅₀ for the different constructs was determined using P24 enzyme-linked immunosorbent assay (ELISA)-based titers.

FIG. 8 is a bar chart showing the titers for glycoprotein particles comprising VLPs, and a VLP bald control, as determined by P24 ELISA and plotted as VLP per ml, as described in Example 2. All the constructs showed comparable levels of production.

FIG. 9 is a bar chart of editing data showing the fold change in EC₅₀ values over the base control glycoprotein (pGP2; set to 1.0) for the glycoprotein particles comprising VLPs, pseudotyped with different glycoproteins, and a VLP bald control, as described in Example 2.

FIG. 10 depicts the plasmids utilized in the creation of an example glycoprotein particle comprising a VLP, with protease cleavage sequence sites indicated by arrows, as described in Example 2.

FIG. 11 depicts the plasmids utilized in the creation of an example glycoprotein particle comprising a VLP, with protease cleavage sequence sites indicated by arrows, as described in Example 2.

FIG. 12 is a graph of editing results of glycoprotein particles comprising VLPs based on HIV (V168) or Alphavirus (V44 and V102) having various incorporated glycoproteins used to edit tdTomato in NPCs in terms of volume of viral-like particles added to treat the cells, as described in Example 3.

FIG. 13 is a bar graph of editing results with glycoprotein particles comprising VLPs based on HIV (V168) or Alphavirus (V44 and V102) having various incorporated glycoproteins used to edit tdTomato in NPCs treated with the indicated volume of viral-like particles, as described in Example 3.

FIG. 14 is a bar graph of editing results with glycoprotein particles comprising VLPs based on HIV (V168) or Alphavirus (V44 and V102) having various incorporated glycoproteins used to edit tdTomato in NPCs treated with the indicated volume of viral-like particles, as described in Example 3.

FIG. 15 is a graph of editing results with glycoprotein particles comprising VLPs based on HIV (V168) having various incorporated glycoproteins (or bald negative control) used to edit tdTomato in NPCs across a range of titered volumes of viral-like particles added to treat the cells, as described in Example 3.

FIG. 16 is a bar graph showing the titers for glycoprotein particles comprising VLPs based on HIV (V168) having various incorporated glycoproteins, determined by P24 ELISA and plotted as VLPs/ml, as described in Example 3. All the constructs showed comparable levels of production.

FIG. 17 is a graph of editing results with glycoprotein particles comprising VLPs based on HIV (V168) having incorporated glycoproteins from rabies variants used to edit tdTomato in NPCs across a range of volumes of VLPs added to treat the cells, as described in Example 3.

FIG. 18 is a bar graph of editing results with glycoprotein particles comprising VLPs based on HIV (V168) having incorporated glycoproteins from rabies variants used to edit tdTomato in NPCs across the indicated volume of viral-like particles added to treat the cells, as described in Example 3.

FIG. 19 is a graph of editing results with glycoprotein particles comprising VLPs based on HIV (V168) having incorporated glycoproteins from Paramyxoviridae, Orthomyxoviridae, and Flaviviridae used to edit tdTomato (TDT) in NPCs across a range of volumes of VLPs added to treat the cells, as described in Example 3.

FIG. 20 is a bar graph of editing results with glycoprotein particles comprising VLPs based on HIV (V168) having various incorporated glycoproteins used to edit tdTomato in NPCs across the indicated volumes of VLPs added to treat the cells, as described in Example 3.

FIG. 21 is a bar graph showing the titers for the glycoprotein particles comprising viral-like constructs based on HIV (V168) having various incorporated glycoproteins, determined by P24 ELISA and plotted as VLP/ml, as described in Example 3. All the constructs showed comparable levels of production.

FIG. 22 is a bar graph of editing results with glycoprotein particles comprising VLPs based on HIV having various incorporated glycoproteins used to edit tdTomato in mouse NPCs, as described in Example 4.

FIG. 23 is a bar graph of editing results with glycoprotein particles comprising VLPs based on HIV having various incorporated glycoproteins used to edit the beta 2 microglobulin (B2M) in human NPCs, as described in Example 4.

FIG. 24 is a bar graph of editing results with glycoprotein particles comprising VLPs based on HIV having various incorporated glycoproteins used to edit the B2M locus in human astrocytes, as described in Example 4.

FIG. 25 is a bar graph of editing results with glycoprotein particles comprising VLPs based on HIV having various incorporated glycoproteins used to edit the B2M locus in human Jurkat cells, as described in Example 4.

### DETAILED DESCRIPTION

Provided herein are glycoprotein particles, i.e., particles comprising a glycoprotein, for the delivery of any payload, including proteins, nucleic acids, small molecules, or combinations thereof, to selectively target cells and/or tissues. As provided herein, the glycoprotein particles of the present disclosure improve cell and tissue tropism, providing targeted delivery of a payload to a cell or tissue of interest. Glycoprotein particle delivery of a payload as provided herein may be for any application, including therapeutic and diagnostic applications. Glycoprotein particles of the present disclosure may comprise any delivery particle including, but not limited to, viral-like particles, lipid nanoparticles, liposomes, exosomes, organic polymer-based particles, inorganic nanoparticles, or any combination thereof.

### I. Definitions

The practice of the present invention employs, unless otherwise indicated, conventional techniques of immunology, biochemistry, chemistry, molecular biology, microbiology, cell biology, genomics and recombinant DNA, which can be found in such standard textbooks as Molecular Cloning: A Laboratory Manual, 3rd Ed. (Sambrook et al., Cold Spring Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that endpoints are included and that each intervening value, to the tenth of the unit of the lower limit unless the context clearly dictates otherwise, between the upper and lower limit of that range and any other stated or intervening value in that stated range, is encompassed. The upper and lower limits of these smaller ranges may independently be included in the smaller ranges, and are also encompassed, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of ordinary skill in the art to which this invention belongs. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise.

It will be appreciated that certain features of the disclosure, which are, for clarity, described in the context of separate embodiments, may also be provided in combination in a single embodiment. In other cases, various features of the disclosure, which are, for brevity, described in the context of a single embodiment, may also be provided separately or in any suitable sub-combination. It is intended that all combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

As used herein, the terms “identity” and “identical,” when referring to a comparison of two sequences, refers to the percentage of exact matching residues in an alignment of a sequence provided herein to a reference sequence, such as an alignment generated by a BLAST algorithm or other alignment algorithms known in the art. Identity may be calculated based on an alignment of a full length sequence provided herein and a full length reference sequence. Identity may also be calculated based on a partial alignment of a sequence provided herein and a reference sequence, if the reference sequence is longer than a sequence provided herein. Identity may also be calculated based on a partial alignment of a sequence provided herein and a reference sequence, if the reference sequence is shorter than a sequence provided herein. Thus, when aligning two sequences, according to the aforementioned, a query sequence “shares at least x % identity to” a subject sequence if in the alignment of the two sequences, at least x % (rounded down) of the residues in the subject sequence are aligned as an exact match to a corresponding residue in the query sequence, wherein the numerator is the number of exact matches and the denominator is the length of the query sequence. In some embodiments, the denominator may alternatively be the length of the query sequence minus any gaps of two or more non-matching residues. Where the subject sequence has variable positions (e.g., residues denoted X), an alignment to any residue in the query sequence is counted as a match.

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

The terms “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence.

The term “naturally-occurring” or “unmodified” or “wild type” as used herein as applied to a nucleic acid, a polypeptide, a cell, or an organism, refers to a nucleic acid, polypeptide, cell, or organism that is found in nature.

As used herein, a “mutation” refers to an insertion, deletion, substitution, duplication, or inversion of one or more amino acids or nucleotides as compared to a wild-type or reference amino acid sequence or to a wild-type or reference nucleotide sequence.

The term “antibody,” as used herein, encompasses various antibody structures, including but not limited to monoclonal antibodies, polyclonal antibodies, multispecific antibodies (e.g., bispecific antibodies), nanobodies, single domain antibodies such as VHH antibodies, and antibody fragments so long as they exhibit the desired antigen-binding activity or immunological activity. Antibodies represent a large family of molecules that include several types of molecules, such as IgD, IgG, IgA, IgM and IgE.

An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2, diabodies, single chain diabodies, linear antibodies, a single domain antibody, a single domain camelid antibody, single-chain variable fragment (scFv) antibody molecules, and multispecific antibodies formed from antibody fragments.

As used herein, “treatment” or “treating,” are used interchangeably herein and refer to an approach for obtaining beneficial or desired results, including but not limited to a therapeutic benefit and/or a prophylactic benefit. By therapeutic benefit is meant eradication or amelioration of the underlying disorder or disease being treated. A therapeutic benefit can also be achieved with the eradication or amelioration of one or more of the symptoms or an improvement in one or more clinical parameters associated with the underlying disease such that an improvement is observed in the subject, notwithstanding that the subject may still be afflicted with the underlying disorder.

The terms “therapeutically effective amount” and “therapeutically effective dose”, as used herein, refer to an amount of a drug or a biologic, alone or as a part of a composition, that is capable of having any detectable, beneficial effect on any symptom, aspect, measured parameter or characteristics of a disease state or condition when administered in one or repeated doses to a subject such as a human or an experimental animal. Such effect need not be absolute to be beneficial.

As used herein, “administering” means a method of giving a dosage of a compound (e.g., a composition of the disclosure) or a composition (e.g., a pharmaceutical composition) to a subject.

As used herein a “subject” is a mammal. Mammals include, but are not limited to, domesticated animals, non-human primates, humans, dogs, rabbits, mice, rats and other rodents.

All publications, patents, and patent applications mentioned in this specification are herein incorporated by reference to the same extent as if each individual publication, patent, or patent application was specifically and individually indicated to be incorporated by reference.

### II. Glycoproteins

The present disclosure provides glycoprotein particles, i.e., particles comprising a viral glycoprotein, or fragment thereof, for the delivery of any payload, including proteins, nucleic acids, small molecules, or combinations thereof, to selectively target cells and tissues. In some embodiments, delivery of a payload by a glycoprotein particle may be for a therapeutic and/or diagnostic treatment of a subject in need thereof.

As used herein, the term “tropism” refers to preferential binding and/or entry of a particle (e.g. glycoprotein particle of the disclosure) to certain cells or tissue type(s) and/or preferential interaction with the cell surface of cells of that cell or tissue type that facilitates binding and/or entry of the particle or its payload to the certain cell or tissue type. When the payload of the particle is a nucleic acid encoding protein or RNA sequence, entry can be followed by expression (e.g., transcription and, optionally, translation) of sequences of the nucleic acid. In many enveloped viruses, viral glycoproteins carry out binding to cellular virus receptors and membrane fusion events that mediate entry of the virus into the host cell. Viral glycoproteins thus confer cell or tissue tropism on the viruses from which they are derived. Without wishing to be bound by theory, the glycoproteins described herein, when incorporated into a particle can confer cell or tissue tropism similar to their role in the native virus.

The term “tropism factor” as used herein refers to components integrated into the surface of a glycoprotein particle that provides tropism for a certain cell or tissue type. Examples of tropism factors that may be used in a glycoprotein particle of the disclosure include glycoproteins, or fragments thereof, as well as antibody fragments (e.g., scFv, nanobodies, linear antibodies, etc.), receptors, and ligands to target cell receptors or cell surface markers.

A glycoprotein for use in a glycoprotein particle of the disclosure refers to an envelope virus glycoprotein, or a fragment of a glycoprotein comprising a virion surface domain. Without being held to any theory or mechanism, native glycosylated viral proteins bind preferentially to specific host cell receptor proteins, conferring cell and tissue specific tropism to a virus. Glycoproteins are a major component of the outermost surface of enveloped viruses, and mediate binding of the virus to the host cell. Thus, a glycoprotein particle as provided herein is a particle that comprises a viral glycoprotein that may confer the cell and tissue specific tropism of the virus from which the viral glycoprotein was derived to the glycoprotein particle. In some embodiments, the glycoprotein particle comprises a full-length glycosylated protein, e.g., one or more glycoproteins whose sequences are disclose in Table 1. In other embodiments, the glycoprotein particle comprises a fragment of a glycoprotein comprising a virion surface domain.

Glycans (oligosaccharides) are covalently attached (in a process known as glycosylation) to viral glycoprotein precursors either during or after translation of the glycoprotein precursor in a host cell to create a glycoprotein. Without wishing to be bound by theory, it is thought that glycoprotein tropism is conferred by the virion surface domain of the glycoprotein and the covalently attached glycan. In some embodiments, the glycoprotein particle comprises a glycoprotein fragment comprising the virion surface domain and including the glycosylation amino acid site. Virion surface domains and glycosylation sites of the glycoproteins in Table 1 may be determined by one of skill in the art, e.g., for example with protein databases or using sequence comparison databases. For example, www.uniprot.org provides the domains and glycosylation sites of known proteins: the virion surface domain (SEQ ID NO: 1143) of the single-pass transmembrane VSV-G glycoprotein (SEQ ID NO: 1) is from amino acid 17 to amino acid 474 of the protein, and glycosylation occurs on amino acid 340.

In other embodiments, alignment of an unknown glycoprotein amino acid sequence to a known glycoprotein amino acid sequence may be used to determine the virion surface domain. The person of ordinary skill in the art will be able to use sequence alignment, e.g. using alignment tools such as with www.blast.ncbi.nlm.nih.gov/Blast.cgi, to align a first glycoprotein with an unknown virion domain and/or glycosylation site to a second glycoprotein of single pass transmembrane sequence whose virion domain and/or glycosylation site are known, thereby determining the unknown virion domain and/or glycosylation site of the first glycoprotein. For example, a single pass transmembrane glycoprotein may be aligned with the VSV-G glycoprotein amino acid sequence, or other single pass transmembrane glycoprotein sequences, to determine the virion surface domain of a single-pass transmembrane glycoprotein.

In some embodiments, a glycoprotein particle comprises the virion surface domain (SEQ ID NO: 1147) of the single pass transmembrane MARV glycoprotein (SEQ ID NO: 91); the virion surface domain (SEQ ID NO: 1151) of the single pass transmembrane COCV glycoprotein (SEQ ID NO: 86); and/or the virion surface domain (SEQ ID NO: 1155) of the single pass transmembrane rabies glycoprotein (SEQ ID NO: 21).

In some embodiments, a glycoprotein particle comprises one or more virion surface domains of the multipass WEEV glycoprotein (SEQ ID NO: 64), i.e., SEQ ID NOS: 1159, 1162, and 1165.

In some embodiments, a glycoprotein particle comprises the virion surface domain of measles H (SEQ ID NO: 111), i.e., SEQ ID NO: 1169. In some embodiments, a glycoprotein particle comprises the virion surface domain of measles F (SEQ ID NO: 110), i.e., SEQ ID NO: 1173.

In some embodiments, a glycoprotein particle comprises a glycoprotein sequence comprising a cleavage site, e.g., an enzymatic or photosensitive linker, integrated into the amino acid sequence to result in a glycoprotein particle comprising a fragment of the original glycoprotein. In some embodiments, a glycoprotein particle comprises a glycoprotein fragment that results from non-specific cleavage. In some embodiments, a glycoprotein particle comprises a glycoprotein fragment encoded by a nucleic acid sequence.

As provided herein, glycoprotein particles of the present disclosure may comprise glycoproteins, or virion surface domain fragments thereof, derived from envelope virus. Envelope viruses include but are not limited to: Argentine hemorrhagic fever virus, Australian bat virus, Autographa californica multiple nucleopolyhedrovirus, Avian leukosis virus, baboon endogenous virus, Bolivian hemorrhagic fever virus, Borna disease virus, Breda virus, Bunyamwera virus, Chandipura virus, Chikungunya virus, Crimean-Congo hemorrhagic fever virus, Dengue fever virus, Duvenhage virus, Eastern equine encephalitis virus, Ebola hemorrhagic fever virus, Ebola Zaire virus, enteric adenovirus, Ephemerovirus, Epstein-Bar virus (EBV), European bat virus 1, European bat virus 2, Fug Synthetic gP Fusion, Gibbon ape leukemia virus, Hantavirus, Hendra virus, hepatitis A virus, hepatitis B virus, hepatitis C virus, hepatitis D virus, hepatitis E virus, hepatitis G Virus (GB virus C), herpes simplex virus type 1, herpes simplex virus type 2, human cytomegalovirus (HHV5), human foamy virus, human herpesvirus (HHV), human Herpesvirus 7, human herpesvirus type 6, human herpesvirus type 8, human immunodeficiency virus 1 (HIV-1), human metapneumovirus, human T-lymphotro pic virus 1, influenza A, influenza B, influenza C virus, Japanese encephalitis virus, Kaposi's sarcoma-associated herpesvirus (HHV8), Kaysanur Forest disease virus, La Crosse virus, Lagos bat virus, Lassa fever virus, lymphocytic choriomeningitis virus (LCMV), Machupo virus, Marburg hemorrhagic fever virus, measles virus, Middle eastern respiratory syndrome-related coronavirus, Mokola virus, Moloney murine leukemia virus, monkey pox, mouse mammary tumor virus, mumps virus, murine gammaherpesvirus, Newcastle disease virus, Nipah virus, Nipah virus, Norwalk virus, Omsk hemorrhagic fever virus, papilloma virus, parvovirus, pseudorabies virus, Quaranfil virus, rabies virus, RD114 Endogenous Feline Retrovirus, respiratory syncytial virus (RSV), Rift Valley fever virus, Ross River virus, rRotavirus, Rous sarcoma virus, rubella virus, Sabia-associated hemorrhagic fever virus, SARS-associated coronavirus (SARS-COV), Sendai virus, Tacaribe virus, Thogotovirus, tick-borne encephalitis causing virus, varicella zoster virus (HHV3), varicella zoster virus (HHV3), variola major virus, variola minor virus, Venezuelan equine encephalitis virus, Venezuelan hemorrhagic fever virus, vesicular stomatitis virus (VSV), Vesiculovirus, West Nile virus, western equine encephalitis virus, and Zika Virus. Non-limiting examples of envelope virus glycoprotein amino acid sequences are provided in Table 1.

TABLE 1
Exemplary Glycoprotein sequences

| | SEQ |
|---|---|
| Virus | ID NO |
| Vesicular Stomatitis Virus (VSV-G) | 1 |
| Human Immunodeficiency Virus | 2 |
| Avian leukosis virus | 3 |
| Rous Sarcoma Virus | 4 |
| Mouse mammary tumor virus | 5 |
| Human T-lymphotropic virus 1 (HTLV1) | 6 |
| RD114 Endogenous Feline Retrovirus | 7 |
| Gibbon ape leukemia virus (GALV) | 8 |
| Moloney Murine leukemia virus | 9 |
| Baboon Endogenous Virus (BABV) | 10 |
| Human Foamy Virus | 11 |
| Psuedorabies virus | 12 |
| Psuedorabies virus | 13 |
| Psuedorabies virus | 14 |
| Psuedorabies virus | 15 |
| Herpes simplex virus 1 (HHV1) | 16 |
| Herpes simplex virus 1 (HHV1) | 17 |
| Herpes simplex virus 1 (HHV1) | 18 |
| Herpes simplex virus 1 (HHV1) | 19 |
| Hepatitis C Virus | 20 |
| Rabies Virus | 21 |
| Mokola Virus | 22 |
| Measles Virus | 23 |
| Measles Virus | 24 |
| Ebola Zaire Virus | 25 |
| Dengue | 26 |
| Zika virus | 27 |
| West Nile Virus | 28 |
| Japanese Encephalitis Virus | 29 |
| Hepatitis G Virus | 30 |
| Mumps Virus F | 31 |
| Mumps Virus HN | 32 |
| Sendai Virus F | 33 |
| Sendai Virus HN | 34 |
| AcMNPV gp64 | 35 |
| Ross River Virus | 36 |
| Codon optimized rabies virus | 37 |
| Rabies virus (strain Nishigahara RCEH) (RABV) | 38 |
| Rabies virus (strain India) (RABV) | 39 |
| Rabies virus (strain CVS-11) (RABV) | 40 |
| Rabies virus (strain ERA) (RABV) | 41 |
| Rabies virus (strain SAD B19) (RABV) | 42 |
| Rabies virus (strain Vnukovo-32) (RABV) | 43 |
| Rabies virus (strain Pasteur vaccins/PV) (RABV) | 44 |
| Rabies virus (strain PM1503/AVO1) (RABV) | 45 |
| Rabies virus (strain China/DRV) (RABV) | 46 |
| Rabies virus (strain China/MRV) (RABV) | 47 |
| Rabies virus (isolate Human/Algeria/1991) (RABV) | 48 |
| Rabies virus (strain HEP-Flury) (RABV) | 49 |
| Rabies virus (strain silver-haired bat-associated) | 50 |
| (RABV) (SHBRV) | |
| HSV2 gB | 51 |
| HSV2 gD | 52 |
| HSV2 gH | 53 |
| HSV2 gL | 54 |
| Varicella gB | 55 |
| Varicella gK | 56 |
| Varicella gH | 57 |
| Varicella gL | 58 |
| Hepatitis B gL | 59 |
| Hepatitis B gM | 60 |
| Hepatitis B gS | 61 |
| Eastern equine encephalitis virus (EEEV) | 62 |
| Venezuelan equine encephalitis viruses (VEEV) | 63 |
| Western equine encephalitis virus (WEEV) | 64 |
| Semliki Forest virus | 65 |
| Sindbis virus | 66 |
| Chikungunya virus (CHIKV) | 67 |
| Bornavirus BoDV-1 | 68 |
| Tick-borne encephalitis virus (TBEV) | 69 |
| Usutu virus | 70 |
| St. Louis encephalitis virus | 71 |
| Yellow fever virus | 72 |
| Dengue virus 2 | 73 |
| Dengue virus 3 | 74 |
| Dengue virus 4 | 75 |
| Murray Valley encephalitis virus (MVEV) | 76 |
| Powassan virus | 77 |
| H5 Hemagglutinin | 78 |
| H7 Hemagglutinin | 79 |
| N1 Neuraminidase | 80 |
| Canine Distemper Virus | 81 |
| VSAV | 82 |
| ABVV | 83 |
| CARV | 84 |
| CHPV | 85 |
| COCV | 86 |
| VSIV | 87 |
| ISFV | 88 |
| JURV | 89 |
| MSPV | 90 |
| MARV | 91 |
| MORV | 92 |
| VSNJV | 93 |
| PERV | 94 |
| PIRYV | 95 |
| RADV | 96 |
| YBV | 97 |
| VSV CEN AM - 94GUB | 98 |
| VSV South America 85CLB | 99 |
| Nipah Virus | 100 |
| Nipah Virus | 101 |
| Hendra Virus | 102 |
| Hendra Virus | 103 |
| Newcastle disease virus | 104 |
| Newcastle disease virus | 105 |
| RSV f0 | 106 |
| RSV G | 107 |
| Bovine respiratory syncytial virus (strain Rb94) | 108 |
| (BRS) | |
| Murine pneumonia virus (strain 15) (MPV) | 109 |
| Measles virus (strain Edmonston) (MeV) (Subacute | 110 |
| sclerose panencephalitis virus) | |
| Measles virus (strain Edmonston B) (MeV) | 111 |
| (Subacute sclerose panencephalitis virus) | |
| Human respiratory syncytial virus B (strain B1) | 112 |
| Rinderpest virus (strain RBOK) (RDV) | 113 |
| Simian virus 41 (SV41) | 114 |
| Mumps virus (strain Miyahara vaccine) (MuV) | 115 |
| Canine distemper virus (strain Onderstepoort) (CDV) | 116 |
| Human respiratory syncytial virus A (strain Long) | 117 |
| Sendai virus (strain Fushimi) (SeV) | 118 |
| Human respiratory syncytial virus A (strain RSS-2) | 119 |
| Rinderpest virus (strain RBT1) (RDV) | 120 |
| Measles virus (strain Leningrad-16) (MeV) | 121 |
| (Subacute sclerose panencephalitis virus) | |
| Human parainfluenza 2 virus (HPIV-2) | 122 |
| Avian metapneumovirus (isolate Canada goose/ | 123 |
| Minnesota/15a/2001) (AMPV) | |
| Phocine distemper virus (PDV) | 124 |
| Sendai virus (strain Harris) (SeV) | 125 |
| Bovine parainfluenza 3 virus (BPIV-3) | 126 |
| Measles virus (strain Ichinose-B95a) (MeV) | 127 |
| (Subacute sclerose panencephalitis virus) | |
| Human parainfluenza 2 virus (strain Toshiba) | 128 |
| (HPIV-2) | |
| Newcastle disease virus (strain B1-Hitchner/47) | 129 |
| (NDV) | |
| Measles virus (strain Yamagata-1) (MeV) | 130 |
| (Subacute sclerose panencephalitis virus) | |
| Measles virus (strain IP-3-Ca) (MeV) (Subacute | 131 |
| sclerose panencephalitis virus) | |
| Measles virus (strain Edmonston-AIK-C vaccine) | 132 |
| (MeV) (Subacute sclerose panencephalitis virus) | |
| Turkey rhinotracheitis virus (TRTV) | 133 |
| Human parainfluenza 2 virus (strain Greer) | 134 |
| (HPIV-2) | |
| Hendra virus (isolate Horse/Autralia/Hendra/1994) | 135 |
| Human metapneumovirus (strain CAN97-83) (HMPV) | 136 |
| Bovine respiratory syncytial virus (strain | 137 |
| Copenhagen) (BRS) | |
| Sendai virus (strain Z) (SeV) (Sendai virus (strain | 138 |
| HVJ)) | |
| Human parainfluenza 3 virus (strain Wash/47885/57) | 139 |
| (HPIV-3) (Human parainfluenza 3 virus (strain NIH | |
| 47885)) | |
| Mumps virus (strain SBL-1) (MuV) | 140 |
| Measles virus (strain Edmonston-Zagreb vaccine) | 141 |
| (MeV) (Subacute sclerose panencephalitis virus) | |
| Human parainfluenza 1 virus (strain C39) (HPIV-1) | 142 |
| Sendai virus (strain Hamamatsu) (SeV) | 143 |
| Mumps virus (strain RW) (MuV) | 144 |
| Infectious hematopoietic necrosis virus (strain | 145 |
| Oregon69) (IHNV) | |
| Drosophila melanogaster sigma virus (isolate | 146 |
| Drosophila/USA/AP30/2005) (DMelSV) | |
| Hirame rhabdovirus (strain Korea/CA 9703/1997) | 147 |
| (HIRRV) | |
| Sonchus yellow net virus (SYNV) | 148 |
| European bat lyssavirus 1 (strain Bat/Germany/ | 149 |
| RV9/1968) (EBLV1) | |
| Lagos bat virus (LBV) | 150 |
| Duvenhage virus (DUVV) | 151 |
| West Caucasian bat virus (WCBV) | 152 |
| European bat lyssavirus 2 (strain Human/Scotland/ | 153 |
| RV1333/2002) (EBLV2) | |
| Irkut virus (IRKV) | 154 |
| Tupaia virus (isolate Tupaia/Thailand/—/1986) | 155 |
| (TUPV) | |
| Rabies virus (strain ERA) (RABV) | 156 |
| Ovine respiratory syncytial virus (strain WSU | 157 |
| 83-1578) (ORSV) | |
| Human respiratory syncytial virus A (strain | 158 |
| rsb5857) | |
| Piry virus (PIRYV) | 159 |
| Human respiratory syncytial virus A (strain | 160 |
| rsb6190) | |
| Rabies virus (strain SAD B19) (RABV) | 161 |
| Australian bat lyssavirus (isolate Human/AUS/ | 162 |
| 1998) (ABLV) | |
| Rabies virus (strain Vnukovo-32)( RABV) | 163 |
| Aravan virus (ARAV) | 164 |
| Sigma virus | 165 |
| Viral hemorrhagic septicemia virus (strain 07-71) | 166 |
| (VHSV) | |
| Rabies virus (strain Pasteur vaccins/PV) (RABV) | 167 |
| Bovine respiratory syncytial virus (strain Rb94) | 168 |
| (BRS) | |
| Tibrogargan virus (strain CS132) (TIBV) | 169 |
| Infectious hematopoietic necrosis virus (strain | 170 |
| Round Butte) (IHNV) | |
| Human respiratory syncytial virus B (strain 18537) | 171 |
| Adelaide River virus (ARV) | 172 |
| Australian bat lyssavirus (isolate Bat/AUS/1996) | 173 |
| (ABLV) | |
| Bovine ephemeral fever virus (strain BB7721) | 174 |
| (BEFV) | |
| Isfahan virus (ISFV) | 175 |
| Rabies virus (strain silver-haired bat-associated) | 176 |
| (RABV) (SHBRV) | |
| Snakehead rhabdovirus (SHRV) | 177 |
| Infectious hematopoietic necrosis virus (strain | 178 |
| WRAC) (IHNV) | |
| Zaire ebolavirus (strain Kikwit-95) (ZEBOV) | 179 |
| (Zaire Ebola virus) | |
| Sudan ebolavirus (strain Maleo-79) (SEBOV) (Sudan | 180 |
| Ebola virus) | |
| Tai Forest ebolavirus (strain Cote d'Ivoire-94) | 181 |
| (TAFV) (Cote d'Ivoire Ebola virus) | |
| Reston ebolavirus (strain Philippines-96) (REBOV) | 182 |
| (Reston Ebola virus) | |
| Lake Victoria marburgvirus (strain Angola/2005) (MARV) | 183 |
| Zaire ebolavirus (strain Eckron-76) (ZEBOV) (Zaire | 184 |
| Ebola virus) | |
| Reston ebolavirus (strain Reston-89) (REBOV) (Reston | 185 |
| Ebola virus) | |
| Tai Forest ebolavirus (strain Cote d'Ivoire-94) | 186 |
| (TAFV) (Cote d'Ivoire Ebola virus) | |
| Lake Victoria marburgvirus (strain Ozolin-75) (MARV) | 187 |
| (Marburg virus (strain South Africa/Ozolin/1975)) | |
| Zaire ebolavirus (strain Mayinga-76) (ZEBOV) (Zaire | 188 |
| Ebola virus) | |
| Lake Victoria marburgvirus (strain Popp-67) (MARV) | 189 |
| (Marburg virus (strain West Germany/Popp/1967)) | |
| Sudan ebolavirus (strain Boniface-76) (SEBOV) (Sudan | 190 |
| Ebola virus) | |
| Reston ebolavirus (strain Reston-89) (REBOV) (Reston | 191 |
| Ebola virus) | |
| Sudan ebolavirus (strain Human/Uganda/Gulu/2000) | 192 |
| (SEBOV) (Sudan Ebola virus) | |
| Zaire ebolavirus (strain Gabon-94) (ZEBOV) (Zaire | 193 |
| Ebola virus) | |
| Reston ebolavirus (strain Reston-89) (REBOV) | 194 |
| (Reston Ebola virus) | |
| Simian virus 41 (SV41) | 195 |
| Newcastle disease virus (strain D26/76) (NDV) | 196 |
| Xenotropic MuLV-related virus (isolate VP42) (XMRV) | 197 |
| Xenotropic MuLV-related virus (isolate VP62) (XMRV) | 198 |
| Simian immunodeficiency virus (isolate F236/smH4) | 199 |
| (SIV-sm) (Simian immunodeficiency virus sooty mangabey | |
| monkey) | |
| Simian immunodeficiency virus (isolate Mm251) (SIV- | 200 |
| mac) (Simian immunodeficiency virus rhesus monkey) | |
| Simian immunodeficiency virus (isolate GB1) (SIV-mnd) | 201 |
| (Simian immunodeficiency virus mandrill) | |
| Simian immunodeficiency virus (isolate Mm142-83) | 202 |
| (SIV-mac) (Simian immunodeficiency virus rhesus monkey) | |
| Simian immunodeficiency virus (isolate MB66) (SIV-cpz) | 203 |
| (Chimpanzee immunodeficiency virus) | |
| Simian immunodeficiency virus (isolate EK505) (SIV-cpz) | 204 |
| (Chimpanzee immunodeficiency virus) | |
| Feline immunodeficiency virus (strain UK2) (FIV) | 205 |
| Feline immunodeficiency virus (strain San Diego) (FIV) | 206 |
| Feline immunodeficiency virus (isolate Wo) (FIV) | 207 |
| Feline immunodeficiency virus (isolate Petaluma) (FIV) | 208 |
| Feline immunodeficiency virus (strain UK8) (FIV) | 209 |
| Feline immunodeficiency virus (strain UT-113) (FIV) | 210 |
| Mayoro Virus | 211 |
| Barmah Forest Virus | 212 |
| Aura virus | 213 |
| Bebaru Virus | 214 |
| Middleburg virus | 215 |
| Mucambo virus | 216 |
| Ndumu Virus | 217 |
| O'nyong-nyong virus | 218 |
| Pixuna virus | 219 |
| Tonate Virus | 220 |
| Trocara virus | 221 |
| Whataroa virus | 222 |
| Bussuquara virus | 223 |
| Jugra virus | 224 |
| MeV-G | 281 |
| MeV-F | 282 |
| Nipah-G | 283 |
| Nipah-F | 284 |
| Queensland carbovirus | 286 |
| Southwest carbovirus | 287 |
| Mammalian 1 orthobornavirus | 288 |
| Mammalian 2 orthobornavirus | 289 |
| Passeriform 1 orthobornavirus | 290 |
| Passeriform 2 orthobornavirus | 291 |
| Psittaciform 1 orthobornavirus | 292 |
| Waterbird 1 orthobornavirus | 293 |
| Elapid 1 orthobornavirus | 294 |
| Lloviu cuevavirus | 295 |
| Lloviu cuevavirus | 296 |
| Mengla dianlovirus | 297 |
| Bombali ebolavirus | 298 |
| Bundibugyo ebolavirus | 299 |
| Reston ebolavirus | 300 |
| Sudan ebolavirus | 301 |
| Tai Forest ebolavirus | 302 |
| Zaire ebolavirus | 303 |
| Marburg marburgvirus | 304 |
| Xilang striavirus | 305 |
| San Jacinto virus | 306 |
| Sierra Nevada virus | 307 |
| Soybean cyst nematode nyami-like virus | 308 |
| Formica fusca virus 1 | 309 |
| Avian metaavulavirus 2 | 310 |
| Avian metaavulavirus 2 | 311 |
| Avian metaavulavirus 5 | 312 |
| Avian metaavulavirus 5 | 313 |
| Avian metaavulavirus 6 | 314 |
| Avian metaavulavirus 6 | 315 |
| Avian metaavulavirus 7 | 316 |
| Avian metaavulavirus 7 | 317 |
| Avian metaavulavirus 8 | 318 |
| Avian metaavulavirus 8 | 319 |
| Avian metaavulavirus 10 | 320 |
| Avian metaavulavirus 10 | 321 |
| Avian metaavulavirus 11 | 322 |
| Avian metaavulavirus 11 | 323 |
| Avian metaavulavirus 14 | 324 |
| Avian metaavulavirus 14 | 325 |
| Avian metaavulavirus 15 | 326 |
| Avian metaavulavirus 15 | 327 |
| Avian metaavulavirus 20 | 328 |
| Avian metaavulavirus 20 | 329 |
| Avian orthoavulavirus 1 | 330 |
| Avian orthoavulavirus 1 | 331 |
| Avian orthoavulavirus 9 | 332 |
| Avian orthoavulavirus 9 | 333 |
| Avian orthoavulavirus 12 | 334 |
| Avian orthoavulavirus 12 | 335 |
| Avian orthoavulavirus 13 | 336 |
| Avian orthoavulavirus 13 | 337 |
| Avian orthoavulavirus 16 | 338 |
| Avian orthoavulavirus 16 | 339 |
| Avian orthoavulavirus 17 | 340 |
| Avian orthoavulavirus 17 | 341 |
| Avian paraavulavirus 3 | 342 |
| Avian paraavulavirus 3 | 343 |
| Avian paraavulavirus 4 | 344 |
| Avian paraavulavirus 4 | 345 |
| Synodus synodonvirus | 346 |
| Synodus synodonvirus | 347 |
| Salmo aquaparamyxovirus | 348 |
| Salmo aquaparamyxovirus | 349 |
| Reptilian ferlavirus | 350 |
| Reptilian ferlavirus | 351 |
| Cedar henipavirus | 352 |
| Cedar henipavirus | 353 |
| Ghanaian bat henipavirus | 354 |
| Ghanaian bat henipavirus | 355 |
| Hendra henipavirus | 356 |
| Hendra henipavirus | 357 |
| Mojiang henipavirus | 358 |
| Mojiang henipavirus | 359 |
| Nipah henipavirus | 360 |
| Nipah henipavirus | 361 |
| Beilong jeilongvirus | 362 |
| Beilong jeilongvirus | 363 |
| Jun jeilongvirus | 364 |
| Jun jeilongvirus | 365 |
| Lophuromys jeilongvirus 1 | 366 |
| Lophuromys jeilongvirus 1 | 367 |
| Lophuromys jeilongvirus 2 | 368 |
| Lophuromys jeilongvirus 2 | 369 |
| Myodes jeilongvirus | 370 |
| Myodes jeilongvirus | 371 |
| Tailam jeilongvirus | 372 |
| Tailam jeilongvirus | 373 |
| Canine morbillivirus | 374 |
| Canine morbillivirus | 375 |
| Cetacean morbillivirus | 376 |
| Cetacean morbillivirus | 377 |
| Feline morbillivirus | 378 |
| Feline morbillivirus | 379 |
| Measles morbillivirus | 380 |
| Measles morbillivirus | 381 |
| Phocine morbillivirus | 382 |
| Phocine morbillivirus | 383 |
| Rinderpest morbillivirus | 384 |
| Rinderpest morbillivirus | 385 |
| Small ruminant morbillivirus | 386 |
| Small ruminant morbillivirus | 387 |
| Mossman narmovirus | 388 |
| Mossman narmovirus | 389 |
| Myodes narmovirus | 390 |
| Myodes narmovirus | 391 |
| Nariva narmovirus | 392 |
| Nariva narmovirus | 393 |
| Tupaia narmovirus | 394 |
| Tupaia narmovirus | 395 |
| Bovine respirovirus 3 | 396 |
| Bovine respirovirus 3 | 397 |
| Caprine respirovirus 3 | 398 |
| Caprine respirovirus 3 | 399 |
| Human respirovirus 1 | 400 |
| Human respirovirus 1 | 401 |
| Human respirovirus 3 | 402 |
| Human respirovirus 3 | 403 |
| Murine respirovirus | 404 |
| Murine respirovirus | 405 |
| Porcine respirovirus 1 | 406 |
| Porcine respirovirus 1 | 407 |
| Squirrel respirovirus | 408 |
| Squirrel respirovirus | 409 |
| Salem salemvirus | 410 |
| Salem salemvirus | 411 |
| Human orthorubulavirus 2 | 412 |
| Human orthorubulavirus 2 | 413 |
| Human orthorubulavirus 4 | 414 |
| Human orthorubulavirus 4 | 415 |
| Mammalian orthorubulavirus 5 | 416 |
| Mammalian orthorubulavirus 5 | 417 |
| Mapuera orthorubulavirus | 418 |
| Mapuera orthorubulavirus | 419 |
| Mumps orthorubulavirus | 420 |
| Mumps orthorubulavirus | 421 |
| Porcine orthorubulavirus | 422 |
| Porcine orthorubulavirus | 423 |
| Simian orthorubulavirus | 424 |
| Simian orthorubulavirus | 425 |
| Achimota pararubulavirus 1 | 426 |
| Achimota pararubulavirus 1 | 427 |
| Achimota pararubulavirus 2 | 428 |
| Achimota pararubulavirus 2 | 429 |
| Menangle pararubulavirus | 430 |
| Menangle pararubulavirus | 431 |
| Sosuga pararubulavirus | 432 |
| Sosuga pararubulavirus | 433 |
| Teviot pararubulavirus | 434 |
| Teviot pararubulavirus | 435 |
| Tioman pararubulavirus | 436 |
| Tioman pararubulavirus | 437 |
| Tuhoko pararubulavirus 1 | 438 |
| Tuhoko pararubulavirus 1 | 439 |
| Tuhoko pararubulavirus 2 | 440 |
| Tuhoko pararubulavirus 2 | 441 |
| Tuhoko pararubulavirus 3 | 442 |
| Tuhoko pararubulavirus 3 | 443 |
| Cynoglossus cynoglossusvirus | 444 |
| Cynoglossus cynoglossusvirus | 445 |
| Hoplichthys hoplichthysvirus | 446 |
| Avian metapneumovirus | 447 |
| Avian metapneumovirus | 448 |
| Human metapneumovirus | 449 |
| Human metapneumovirus | 450 |
| Bovine orthopneumovirus | 451 |
| Bovine orthopneumovirus | 452 |
| Human orthopneumovirus | 453 |
| Human orthopneumovirus | 454 |
| Murine orthopneumovirus | 455 |
| Murine orthopneumovirus | 456 |
| Arboretum almendravirus | 457 |
| Menghai almendravirus | 458 |
| Puerto Almendras almendravirus | 459 |
| Santabarbara arurhavirus | 460 |
| Xiburema arurhavirus | 461 |
| Bahia barhavirus | 462 |
| Muir barhavirus | 463 |
| Caligus caligrhavirus | 464 |
| Lepeophtheirus caligrhavirus | 465 |
| Salmonlouse caligrhavirus | 466 |
| Curionopolis curiovirus | 467 |
| Iriri curiovirus | 468 |
| Itacaiunas curiovirus | 469 |
| Rochambeau curiovirus | 470 |
| Adelaide River ephemerovirus | 471 |
| Berrimah ephemerovirus | 472 |
| Bovine fever ephemerovirus | 473 |
| Hayes ephemerovirus | 474 |
| Kent ephemerovirus | 475 |
| Kimberley ephemerovirus | 476 |
| Koolpinyah ephemerovirus | 477 |
| Kotonkan ephemerovirus | 478 |
| Obodhiang ephemerovirus | 479 |
| Puchong ephemerovirus | 480 |
| Yata ephemerovirus | 481 |
| Flanders hapavirus | 482 |
| Gray Lodge hapavirus | 483 |
| Hart Park hapavirus | 484 |
| Holmes hapavirus | 485 |
| Joinjakaka hapavirus | 486 |
| Kamese hapavirus | 487 |
| La Joya hapavirus | 488 |
| Landjia hapavirus | 489 |
| Manitoba hapavirus | 490 |
| Marco hapavirus | 491 |
| Mosqueiro hapavirus | 492 |
| Mossuril hapavirus | 493 |
| Ord River hapavirus | 494 |
| Parry Creek hapavirus | 495 |
| Wongabel hapavirus | 496 |
| Barur ledantevirus | 497 |
| Bughendera ledantevirus | 498 |
| Fikirini ledantevirus | 499 |
| Fukuoka ledantevirus | 500 |
| Kanyawara ledantevirus | 501 |
| Kern Canyon ledantevirus | 502 |
| Keuraliba ledantevirus | 503 |
| Kolente ledantevirus | 504 |
| Kumasi ledantevirus | 505 |
| Le Dantec ledantevirus | 506 |
| Mount Elgon bat ledantevirus | 507 |
| Nkolbisson ledantevirus | 508 |
| Oita ledantevirus | 509 |
| Vaprio ledantevirus | 510 |
| Wuhan ledantevirus | 511 |
| Yongjia ledantevirus | 512 |
| Lonestar lostrhavirus | 513 |
| Aravan lyssavirus | 514 |
| Australian bat lyssavirus | 515 |
| Bokeloh bat lyssavirus | 516 |
| Duvenhage lyssavirus | 517 |
| European bat 1 lyssavirus | 518 |
| European bat 2 lyssavirus | 519 |
| Gannoruwa bat lyssavirus | 520 |
| Ikoma lyssavirus | 521 |
| Irkut lyssavirus | 522 |
| Khujand lyssavirus | 523 |
| Lagos bat lyssavirus | 524 |
| Lleida bat lyssavirus | 525 |
| Mokola lyssavirus | 526 |
| Rabies lyssavirus | 527 |
| Shimoni bat lyssavirus | 528 |
| Taiwan bat lyssavirus | 529 |
| West Caucasian bat lyssavirus | 530 |
| Culex ohlsrhavirus | 531 |
| Ohlsdorf ohlsrhavirus | 532 |
| Anguillid perhabdovirus | 533 |
| Perch perhabdovirus | 534 |
| Sea trout perhabdovirus | 535 |
| Minto sawgrhavirus | 536 |
| Sawgrass sawgrhavirus | 537 |
| Capitata sigmavirus | 538 |
| Drosophila affinis sigmavirus | 539 |
| Chaco sripuvirus | 540 |
| sripur sripuvirus | 541 |
| Charleville sripuvirus | 542 |
| Garba sunrhavirus | 543 |
| Kwatta sunrhavirus | 544 |
| Sunguru sunrhavirus | 545 |
| Walkabout sunrhavirus | 546 |
| Bas-Congo tibrovirus | 547 |
| Beatrice Hill tibrovirus | 548 |
| Coastal Plains tibrovirus | 549 |
| Ekpoma 1 tibrovirus | 550 |
| Ekpoma 2 tibrovirus | 551 |
| Sweetwater Branch tibrovirus | 552 |
| Tibrogargan tibrovirus | 553 |
| Durham tupavirus | 554 |
| Klamath tupavirus | 555 |
| Tupaia tupavirus | 556 |
| Alagoas vesiculovirus | 557 |
| Carajas vesiculovirus | 558 |
| Chandipura vesiculovirus | 559 |
| Cocal vesiculovirus | 560 |
| Indiana vesiculovirus | 561 |
| Isfahan vesiculovirus | 562 |
| Jurona vesiculovirus | 563 |
| Malpais Spring vesiculovirus | 564 |
| Maraba vesiculovirus | 565 |
| Morreton vesiculovirus | 566 |
| New Jersey vesiculovirus | 567 |
| Perinet vesiculovirus | 568 |
| Piry vesiculovirus | 569 |
| Radi vesiculovirus | 570 |
| Yug Bogdanovac vesiculovirus | 571 |
| Zahedan zarhavirus | 572 |
| Reptile sunshinevirus 1 | 573 |

In some embodiments, a glycoprotein particle of the disclosure comprises a glycoprotein comprising an amino acid sequence of SEQ ID NOS: 1-224, 281-284, and 286-573 as set forth in Table 1, or a sequence having at least 70%, at least 80%, at least 85%, at least 90%, at least 95%, at least 96%, at least 97%, at least 98%, at least 99% sequence identity thereto. In some embodiments, a glycoprotein particle of the disclosure comprises a glycoprotein comprising an amino acid sequence of SEQ ID NOS: 1-224, 281-284, and 286-573 as set forth in Table 1.

In some embodiments, a glycoprotein particle of the disclosure comprises a glycoprotein fragment comprising a virion surface domain of a glycoprotein of SEQ ID NOS: 1-224, 281-284, and 286-573 as set forth in Table 1, or a sequence having at least 70%, at least 80%, at least 85%, at least 90%, at least 95%, at least 96%, at least 97%, at least 98%, at least 99%, sequence identity thereto. In some embodiments, a glycoprotein particle of the disclosure comprises a glycoprotein fragment comprising a virion surface domain of a glycoprotein of SEQ ID NOS: 1-224, 281-284, and 286-573 as set forth in Table 1.

In some embodiments, a glycoprotein particle of the disclosure comprises a combination of two or more full-length glycoproteins to confer tropism of the particle to a target cell. In some embodiments, a glycoprotein particle of the disclosure comprises a combination of two or more glycoprotein fragments to confer tropism of the particle to a target cell. In some embodiments, a glycoprotein particle of the disclosure comprises a combination of one or more full-length glycoproteins and one or more glycoprotein fragments to confer tropism of the particle to a target cell.

### III. Particles

The disclosure provides glycoprotein particles comprising viral-like particles, lipid nanoparticles, liposomes, exosomes, organic polymer-based particles, inorganic nanoparticles, or any combination thereof. A glycoprotein particle as provided herein is designed to deliver one or more payloads to a target cell or tissue.

### Viral-Like Particles (VLP)

In some embodiments, a glycoprotein particle of the disclosure comprises a viral-like particle (VLP), wherein the VLP is a non-replicating, self-assembling, non-naturally occurring multicomponent structure comprising viral proteins and polyproteins, such as, but not limited to, a capsid, coat, shell, and/or a lipid layer surrounding the VLP derived from the host packaging cell.

In some embodiments, the VLP is derived from a retrovirus, adeno-associated virus (AAV), lentivirus, adenovirus, or herpes simplex virus. In some embodiments, the VLP components may be selected from a Retroviridae virus, e.g. an Orthoretrovirinae virus or a Spumaretrovirinae virus. In some embodiments, the Orthoretrovirinae virus is selected from the group consisting of an Alpharetrovirus, Betaretrovirus, Deltaretrovirus, Epsilonretrovirus, Gammaretrovirus, and Lentivirus. In some embodiments, the Spumaretrovirinae virus is selected from the group consisting of Bovispumavirus, Equispumavirus, Felispumavirus, Prosimiispumavirus, Simiispumavirus, or Spumavirus.

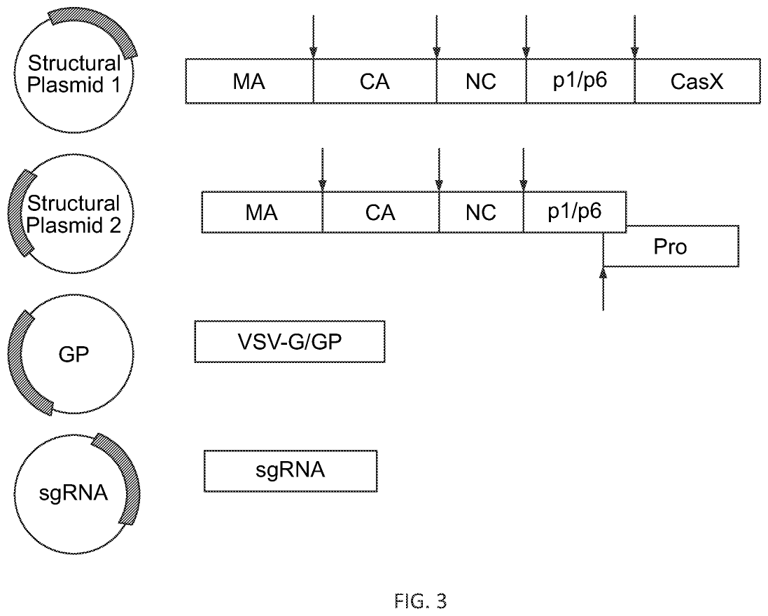

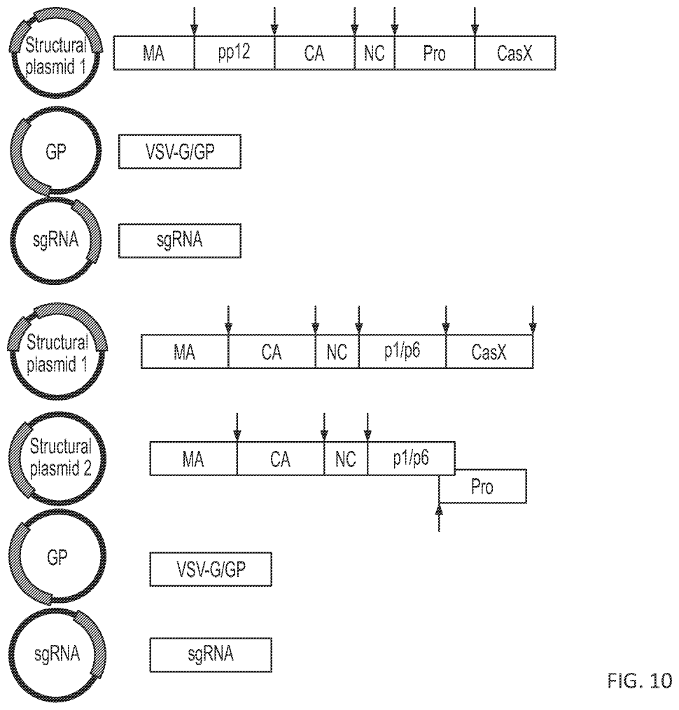

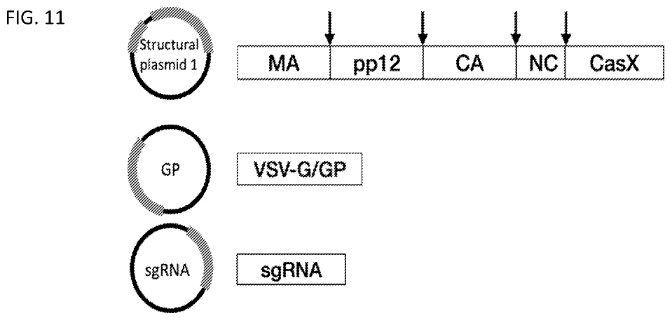

In some embodiments, a glycoprotein particle comprising a VLP is capable of self-assembling when one or more nucleic acids encoding the components of the VLP and the glycoprotein are introduced into a eukaryotic host packaging cell and are expressed. A payload may also be expressed in the host cell and encapsidated within the VLP upon self-assembly. Exemplary embodiments are provided in the Examples, and shown in FIGS. 3, 10, and 11.

Exemplary VLPs are known in the art, and are described in WO2021113772A1, the contents of which are incorporated by reference in their entirety herein.

In some embodiments, the VLP is derived from a retrovirus, or comprises retroviral proteins. The major structural component of retroviruses is the polyprotein Gag, which also typically contain protease cleavage sites that, upon action by the viral protease, processes the Gag into subcomponents that, in the case of the replication of the source virus, then self-assemble in the host cell to make the core inner shell of the virus. The expression of Gag alone is sufficient to mediate the assembly and release of viral-like particles (VLPs) from host cells. Gag proteins from all retroviruses contain an N-terminal membrane-binding matrix (MA) domain, a capsid (CA) domain (with two subdomains), and a nucleocapsid (NC) domain that are structurally similar across retroviral genera but differ greatly in sequence. Outside these core domains, Gag proteins vary among retroviruses, and other linkers and domains may be present (Shur, F., et al. The Structure of Immature Virus-Like Rous Sarcoma Virus Gag Particles Reveals a Structural Role for the plO Domain in Assembly. J Virol. 89(20): 10294 (2015)). The assembly pathway of Gag into immature particles in the host cell is mediated by interactions between MA (which is responsible for targeting Gag polyprotein to the plasma membrane), between NC and RNA, and between CA domains (which, in the context of the present disclosure, can assemble into a VLP capsid). For most retrovirus genera, assembly takes place on the plasma membrane, but for betaretroviruses the particles are assembled in the cytoplasm and then transported to the plasma membrane. In the context of the retroviruses, concomitant with, or shortly after, particle release, cleavage of Gag by the viral protease (PR) gives rise to separate MA, CA, and NC proteins, inducing a rearrangement of the internal viral structure, with CA forming the shell of the mature viral core. Full proteolytic cleavage of Gag into its individual domains is necessary for virus infectivity for the native viruses. However, it has been discovered that for self-assembly of VLP within a host cell comprising retroviral components that are then capable of being taken up by target cells and delivering the therapeutic payload, the VLP does not require cleavage of Gag; hence the omission of a protease and cleavage sites is optional.

The Retroviridae family of viruses have different subfamilies, including Orthoretrovirinae, Spumaretrovirinae, and unclassified Retroviridae, all of which are envisaged giving rise to components of the VLP of the instant disclosure. Many retroviruses cause serious diseases in humans, other mammals, and birds. Human retroviruses include Human Immunodeficiency Virus 1 (HIV-1) and HIV-2, the cause of the disease AIDS, and human T-lymphotropic virus (HTLV) also cause disease in humans. The subfamily Orthoretrovirinae include the genera Alpharetrovirus, Betaretrovirus, Deltaretrovirus, Epsilonretrovirus, Gammaretrovirus, and Lentivirus. Members of Alpharetrovirus, including Avian leukosis virus and Rous sarcoma virus, can cause sarcomas, tumors, and anemia of wild and domestic birds. Examples of Betaretrovirus include mouse mammary tumor virus, Mason-Pfizer monkey virus, and enzootic nasal tumor virus. Examples of Deltaretrovirus include the bovine leukemia virus and the human T-lymphotropic viruses. Members of Epsilonretrovirus include Walleye dermal sarcoma virus, and Walleye epidermal hyperplasia virus 1 and 2. Members of Gammaretrovirus include murine leukemia virus, Maloney murine leukemia virus, and feline leukemia virus, as well as viruses that infect other animal species. Lentivirus is a genus of retroviruses that cause chronic and deadly diseases, including HIV-1 and HIV-2, the cause of the disease AIDS, and also includes Simian immunodeficiency virus. The subfamily Spumaretrovirinae include the genera Bovispumavirus, Equispumavirus, Felispumavirus, Prosimiispumavirus, Simiispumavirus, and Spumavirus. Members of the Retroviridae have provided valuable research tools in molecular biology, and, in the context of the present disclosure, can used in the generation of VLP. The retroviral-derived structural components of VLP can be derived from each of the genera of Retroviridae, and that the resulting VLP are capable self-assembly in a host cell and encapsidating (or encompassing) therapeutic payloads.

In some embodiments, the VLP are pseudotyped, which as used herein, refers to viral envelope proteins in a viral-like particle that have been substituted with those of another virus possessing preferable characteristics, e.g., preferable tropism. For example, HIV can be pseudotyped with vesicular stomatitis virus G-protein (VSV-G) envelope proteins, which allows HIV to infect a wider range of cells.

In some embodiments, the glycoprotein particle comprises recombinant adenovirus and/or adeno-associated virus (AAV) proteins. In some embodiments, the glycoprotein particle comprises or consists of a modified AAV viral vector, e.g., an adenovirus dodecahedron or recombinant adenovirus conglomerate.

AAV-mediated delivery systems are known in the art, and are described, for example, in PCT/US2021/062714, the contents of which are incorporated by reference in their entirety herein.

Being naturally replication-defective and capable of transducing nearly every cell type in the human body, AAV, and modified AAV, represent suitable vectors for therapeutic use in gene therapy or vaccine delivery. Typically, when producing a recombinant AAV, the sequence between the two inverted terminal repeats (ITRs) is replaced with one or more sequences of interest (e.g., a transgene), and the Rep and Cap sequences are provided in trans, making the ITRs the only viral DNA that remains in the vector. The resulting recombinant AAV vector genome construct comprises two cis-acting 130 to 145-nucleotide ITRs flanking an expression cassette encoding the transgene sequences of interest, providing at least 4.7 kb or more for packaging of foreign DNA that can include a transgene, one or more promoters and accessory elements, such that the total size of the vector is below 5 to 5.2 kb, which is compatible with packaging within the AAV capsid (it being understood that as the size of the construct exceeds this threshold, the packaging efficiency of the vector decreases). The transgene may encode a therapeutic or diagnostic payload, as described herein.

By “adeno-associated virus inverted terminal repeats” or “AAV ITRs” is meant the art recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome.

The nucleotide sequences of AAV ITR regions are known in the art. See, for example Kotin, R. M. (1994) Human Gene Therapy 5:793-801; Berns, K. I. “Parvoviridae and their Replication” in Fundamental Virology, 2nd Edition, (B. N. Fields and D. M. Knipe, eds.). As used herein, an AAV ITR need not have the wild-type nucleotide sequence depicted, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV6, AAV7, AAV8, AAV9, AAV10, AAV11, AAV12, AAV 44.9, AAV-Rh74, and AAVRh10, and modified capsids of these serotypes. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Use of AAV serotypes for integration of heterologous sequences into a host cell is known in the art (see, e.g., WO2018195555A1 and US20180258424A1, incorporated by reference herein). In one particular embodiment, the ITRs are derived from serotype AAV1. In another particular embodiment, the ITRs are derived from serotype AAV2, including the 5′ ITR having sequence

(SEQ ID NO: 1176)
CCTGCAGGCAGCTGCGCGCTCGCTCGCTCACTGAGGCCGCCCGGGCGTC
GGGCGACCTTTGGTCGCCCGGCCTCAGTGAGCGAGCGAGCGCGCAGAGA
GGGAGTGGCCAACTCCATCACTAGGGGTTCCT

and the 3′ ITR having sequence

(SEQ ID NO: 1177)
AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAGCTGCCTGCAGG.

By “AAV rep coding region” is meant the region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome.

By “AAV cap coding region” is meant the region of the AAV genome which encodes the capsid proteins VP1, VP2, and VP3, or functional homologues thereof. These Cap expression products supply the packaging functions which are collectively required for packaging the viral genome.

Glycoprotein particles of the disclosure may be AAV vectors, modified AAV vectors, or comprise any of the AAV proteins or genomes as described herein.

### Lipid Nanoparticles (LNP)

In some embodiments, a glycoprotein particle of the disclosure comprises a lipid-containing particle, e.g., a liposome, a liposomal nanoparticle (LNP), a cationic lipid containing particle, or an exosome. A glycoprotein particle of the disclosure comprising an LNP may comprise a diversity of lipids to confer stability and/or specificity to the LNP. In some embodiments, a glycoprotein particle comprises an LNP comprising ionizable lipids, wherein the ionizable lipids comprise one or more of N-[1-(2,3-dioleyloxy)propyl]-N,N,N-trimethylammonium chloride (DOTMA), 5-carboxyspermylglycinedioctadecylamide (DOGS), 2,3-dioleyloxy-N-[2(spermine-carboxamido)ethyl]-N,N-dimethyl-1-propanaminium (DOSPA), 1,2-Dioleoyl-3-Dimethylammonium-Propane (DODAP), and/or 1,2-Dioleoyl-3-Trimethylammonium-Propane (DOTAP), or variations thereof. An LNP may also comprise one or more of 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane or (DLenDMA), N-dioleyl-N,N-dimethylammonium chloride (DODAC), or variations thereof. In other embodiments, an LNP may comprise a ionizable lipid of XTC (2,2-Dilinoleyl-4-dimethylaminoethyl-[1,3]-dioxolane), MC3 (((6Z,9Z,28Z,31Z)-heptatriaconta-6,9,28,31-tetraen-19-yl 4-(dimethylamino)butanoate), ALNY-100 ((3aR,5s,6aS)—N,N-dimethyl-2,2-di((9Z,12Z)-octadeca-9,12-dienyl)tetrahydro-3aH-cyclopenta[d] [1,3]dioxol-5-amine)), NC98-5 (4,7,13-tris(3-oxo-3-(undecylamino)propyl)-N1,N16-diundecyl-4,7,10,13-tetraazahexadecane-1,16-diamide), or variants thereof.

In some embodiments, a glycoprotein particle of the disclosure comprises an LNP comprising additional lipids that stabilize the particle, wherein the stabilizing lipids are selected from one more of: distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or variants thereof.

In some embodiments, a glycoprotein particle of the disclosure comprises an LNP comprising one or more phosphatidyl lipids, for example, the phosphatidyl compounds (e.g., phosphatidylglycerol, phosphatidylcholine, phosphatidylserine and phosphatidylethanolamine). In some embodiments, the LNP comprises sphingolipids, for example but not limited to, sphingosine, ceramide, sphingomyelin, cerebroside and ganglioside.

In some embodiments, a glycoprotein particle of the disclosure comprises an LNP comprising one or more cholesterol-based lipids. A cholesterol-based lipid may include but is not limited to: PEGylated cholesterol, DC-Choi (N,N-dimethyl-N-ethylcarboxamidocholesterol), 1,4-bis(3-N-oleylamino-propyl)piperazine.

In some embodiments, the addition of PEGylated lipids in a glycoprotein particle comprising an LNP protects an LNP from immune targeting. In some embodiments, lipids modified with other hydrophilic molecules may be substituted for PEGylated lipids.

Glycoproteins of the disclosure can be attached to LNP using any methods known in the art, described in more detail below.

### Organic Polymer-Based Particles

In some embodiments, a glycoprotein particle of the disclosure comprises a particle comprising organic polymers, alone or in combination with other particles, as described herein. A glycoprotein particle comprising organic polymers may comprise, for example, one or more of polyacrylates, polyalkylcyanoacrylates, polylactide, polylactide-polyglycolide copolymers, polycaprolactones, dextran, albumin, gelatin, alginate, collagen, chitosan, cyclodextrins, protamine, polyethylene glycol (PEG)-modified (PEGylated) protamine, poly-D-lysine (PLL), PEGylated PLL, polyethylenimine (PEI), or poly (lactic-co-glycolic acid) (PLGA).

### Inorganic Nanoparticles

In some embodiments, a glycoprotein particle of the disclosure comprises an inorganic nanoparticle, e.g., comprising gold, iron, calcium phosphate and/or silica. Inorganic nanoparticles may exhibit radioactive and/or plasmonic properties, and are thus particularly suitable for diagnostics and imaging. In some embodiments, a glycoprotein particle of the disclosure comprises quantum dots comprising semiconducting materials, e.g., silicon, for use in imaging and diagnostics.

In some embodiments, a glycoprotein particle comprises a microparticle. A microparticle as used herein refers to a particle of matter with a diameter between about 1 and about 1000 μm in size. Microparticles are include a variety of materials, including ceramics, glass, polymers, and metals. In some embodiments, the particle is a polymeric microparticle comprising a glycoprotein of the disclosure. In some embodiments, the polymeric microparticle comprises a polymer micelle.

In some embodiments, a glycoprotein particle comprises a nanoparticle. A nanoparticle as used herein refers to a particle of matter with a diameter between about 1 nm to about 100 nm, optionally from about 500 nm to about 1 μm. Non-limiting examples include fullerenes, metals, biological polymers, dendrimers, quantum dots, lipids, nucleic acid vectors, viral-like-particles, or any combination thereof. In some embodiments, the nanoparticle is a lipid particle (including for example, cationic lipids, non-cationic lipids, cholesterol-based lipids, and PEG-modified lipids). In some embodiments, the nanoparticle is a viral-like nanoparticle (including for example viral structural proteins, lipids, and/or carbohydrates). In some embodiments, the particle is a polymeric nanoparticle, e.g., a nanocapsule or a nanosphere. In some embodiments, the polymeric nanoparticle comprises a polymer micelle.

In some embodiments, a glycoprotein particle comprises one or more of fullerenes, metals, biological polymers, dendrimers, quantum dots, lipids, nucleic acid vectors, and viral structural proteins.

### IV. Payloads

Glycoprotein particles of the disclosure can be configured for delivery of any type of payload including, but not limited to, proteins, nucleic acids, small molecules and combinations thereof. In some embodiments, glycoprotein particle delivery of a payload is for therapeutic and/or diagnostic use. For example, VLPs, LNPs, and organic polymer-based particles can be used to deliver therapeutic payloads for the treatment of a variety of diseases, while inorganic nanoparticles can be used in diagnostic imaging.

Glycoprotein particles of the disclosure can be configured to deliver a diversity of protein-based therapeutics, including, but not limited to cytokines (e.g., interferons (IFNs) α, β, and γ, TNF-α, G-CSF, GM-CSF)), interleukins (e.g., IL-1 to IL-40), growth factors (e.g., VEGF, PDGF, IGF-1, EGF, and TGF-β), enzymes, receptors, microproteins, hormones (e.g., growth hormone, insulin), erythropoietin, RNAse, DNAse, blood clotting factors (e.g. FVII, FVIII, FIX, FX), anticoagulants, bone morphogenetic proteins, engineered protein scaffolds, thrombolytics (e.g., streptokinase, tissue plasminogen activator, plasminogen, and plasmid), antibodies, antibody fragments, antibody fusion proteins, CRISPR proteins (Class 1 and Class 2 Type II, Type V, or Type VI), transcription factors, transposons, reverse transcriptases, viral interferon antagonists, tick proteins, as well as engineered proteins such as anti-cancer modalities or biologics intended to treat diseases such as neurologic, metabolic, cardiovascular, liver, renal, or endocrine diseases and disorders, or any combination of the foregoing. In some embodiments, the glycoprotein particle comprising a CRISPR protein payload comprises a Class 2, Type V CRISPR/Cas protein amino acid sequence of one or more of CasX, Cas12a (Cpf1), Cas12b (C2c1), Cas12c (C2c3), Cas12d (CasY), Cas12e (CasX), Cas12f, Cas12g, Cas12h, Cas12i, Cas12j, Cas12k, Cas14, and/or CasΦ.

Glycoprotein particles of the disclosure can be configured to deliver nucleic acid payloads, including sequences encoding the foregoing protein therapeutic payloads, as well as single-stranded antisense oligonucleotides (ASOs), double-stranded RNA interference (RNAi) molecules, DNA aptamers, RNA aptamers, nucleic acids utilized in gene therapy (e.g., guide RNAs (gRNAs) utilized in CRISPR systems, and donor templates), microRNAs, ribozymes, RNA decoys, circular RNAs, or any combination of the foregoing.

In some embodiments, the glycoprotein particle comprising a CRISPR protein payload comprises a Class 2, Type II CRISPR/Cas protein. In some embodiments, the Class 2, Type II CRISPR/Cas protein comprises Cas9. In some embodiments, the Cas9 is isolated or derived from S. pyogenes, Staphylococcus aureus, Streptococcus thermophilus, Neisseria meningitidis or Treponema denticola. In some embodiments, the glycoprotein particle further comprises a Cas9 guide RNA (gRNA). A Cas9 gRNA refers to a guide RNA which is capable of binding to the Cas9 protein and targeting the Cas9 protein to a target nucleic acid. Sequences of gRNAs configured to hybridize to their cognate CRISPR/Cas protein, describe supra, will be apparent to persons of ordinary skill in the art.

In some embodiments, the glycoprotein particle comprises a nucleic acid encoding a Class 2, Type II CRISPR/Cas protein, e.g., Cas9. Class 2 systems are distinguished from Class 1 systems in that they have a single, large, multi-domain effector protein. In certain embodiments, the Class 2 system utilized in the payload of the glycoprotein particle is a Type II, Type V, or Type VI system. Each type of Class 2 system is further divided into subtypes. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type VI systems can be divided into 5 subtypes: VI-A, VI-B1, VI-B2, VI-C, and VI-D.

Nucleases of Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V nucleases possess a single RNA-guided RuvC domain-containing effector but no HNH domain, and they recognize T-rich PAM 5′ upstream to the target region on the non-targeted strand, which is different from Cas9 systems which rely on G-rich PAM at 3′ side of target sequences. Type V nucleases generate staggered double-stranded breaks distal to the PAM sequence, unlike Cas9, which generates a blunt end in the proximal site close to the PAM. In addition, Type V nucleases degrade ssDNA in trans when activated by target dsDNA or ssDNA binding in cis. In some embodiments, the Type V nucleases utilized in the embodiments recognize a 5′ TC PAM motif and produce staggered ends cleaved by the RuvC domain. The Type V systems (e.g., Cas12) only contain a RuvC-like nuclease domain that cleaves both strands. Type VI (Cas13) are unrelated to the effectors of Type II and V systems and contain two HEPN domains and target RNA. In some embodiments, the glycoprotein particle comprises a payload comprising a Class 2 Type II CRISPR system, e.g., a Type II-A CRISPR system. In some embodiments, the payload comprises a Type II-B CRISPR system. In some embodiments, the payload comprises a Type II-C1 CRISPR system. In some embodiments, the payload comprises a Type II-C2 CRISPR system.

In some embodiments, the glycoprotein particle comprises a payload comprising a Class 2 Type V system. In some embodiments, the payload comprises a Type V-A CRISPR system. In some embodiments, the payload comprises a Type V-B 1 CRISPR system. In some embodiments, the payload comprises a Type V-B2 CRISPR system. In some embodiments, the payload comprises a Type V-C CRISPR system. In some embodiments, the payload comprises a Type V-D CRISPR system. In some embodiments, the payload comprises a Type V-E CRISPR system. In some embodiments, the payload comprises a Type V-F1 CRISPR system. In some embodiments, the Type V CRISPR system is a V-F1 (V-U3) CRISPR system. In some embodiments, the payload comprises a Type V-F2 CRISPR system. In some embodiments, the payload comprises a Type V-F3 CRISPR system. In some embodiments, the payload comprises a Type a V-G CRISPR system. In some embodiments, the Type V CRISPR system is a V-H CRISPR system. In some embodiments, the Type V CRISPR system is a V-I CRISPR system. In some embodiments, the Type V CRISPR system is a V-K (V-U5) CRISPR system. In some embodiments, the Type V CRISPR system is a V-U1 CRISPR system. In some embodiments, the Type V CRISPR system is a V-U2 CRISPR system. In some embodiments, the Type V CRISPR system is a V-U4 CRISPR system. In some embodiments, the Type V CRISPR system is selected from the group consisting of Cas12a (Cpf1), Cas12b (C2c1), Cas12c (C2c3), Cas12d (CasY), Cas12e (CasX), Cas12f, Cas12g, Cas12h, Cas12i, Cas12j, Cas12k, Cas12l, Cas14, and CasΦ.

In some embodiments the glycoprotein particle comprises a payload comprising a Class 2 Type VI system. In some embodiments, the Type VI CRISPR system is a VI-A CRISPR system. In some embodiments, the Type VI CRISPR system is a VI-B 1 CRISPR system. In some embodiments, the Type VI CRISPR system is a VI-B2 CRISPR system. In some embodiments, the Type VI CRISPR system is a VI-C CRISPR system. In some embodiments, the Type VI CRISPR system is a VI-D CRISPR system. In some embodiments, the glycoprotein particle comprises a payload comprising a Type VI CRISPR system selected from Cas13a (C2c2), Cas13b (Group 29/30), Cas13c, and/or Cas13d.

In some embodiments, a glycoprotein particle is configured to deliver a gene editing pair comprising a CasX variant of Table 2 and a nucleic acid comprising one or more guide RNA scaffolds of Table 3. In some embodiments, a glycoprotein particle is configured to deliver a gene editing pair comprising a CasX variant protein of Table 2 and a gRNA comprising a gRNA scaffold of Table 3, wherein the gRNA comprises a targeting sequence, and wherein the targeting sequence of the gRNA has a sequence complementary to and is able to hybridize with a target nucleic acid sequence. Upon hybridization by the CasX and the gRNA gene editing pair with the target nucleic acid sequence, the CasX protein introduces one or more single-strand breaks or double-strand breaks within or near the target nucleic acid. The target nucleic acid can include sequences that contain regulatory elements, coding regions, or non-coding regions of a gene, and repair of the double or single-stranded break can result in a permanent indel (deletion or insertion) or mutation in a target nucleic acid due to the cell's repair mechanisms such as non-homologous end joining (NHEJ). If the particle also includes a donor template, repair of the break can result in the introduction of a sequence at or near the site of the break via templated repair mechanisms or site-specific insertion methods such as e.g., homology-directed repair (HDR), or homology-independent targeted integration (HITI). Additional cellular repair mechanisms that can be used to introduce changes in target nucleic acids using gene editing systems delivered using the glycoprotein particles described herein include, but are not limited to micro-homology mediated end joining (MMEJ), single strand annealing (SSA) or base excision repair (BER). The permanent change in nucleic acid sequence by the gene editing pair, as delivered by the glycoprotein particle, can result in a corresponding modulation of expression or alteration in the function of a gene product.

TABLE 2
CasX Protein Sequences

| SEQ ID NO | Variant |
|---|---|
| 574 | 119 |
| 575 | 429 |
| 576 | 430 |
| 577 | 431 |
| 578 | 432 |
| 579 | 433 |
| 580 | 434 |
| 581 | 435 |
| 582 | 436 |
| 583 | 437 |
| 584 | 438 |
| 585 | 439 |
| 586 | 440 |
| 587 | 441 |
| 588 | 442 |
| 589 | 443 |
| 590 | 444 |
| 591 | 445 |
| 592 | 446 |
| 593 | 447 |
| 594 | 448 |
| 595 | 449 |
| 596 | 450 |
| 597 | 451 |
| 598 | 452 |
| 599 | 453 |
| 600 | 454 |
| 601 | 455 |
| 602 | 456 |
| 603 | 457 |
| 604 | 458 |
| 605 | 459 |
| 606 | 460 |
| 607 | 278 |
| 608 | 279 |
| 609 | 280 |
| 610 | 285 |
| 611 | 286 |
| 612 | 287 |
| 613 | 288 |
| 614 | 290 |
| 615 | 291 |
| 616 | 293 |
| 617 | 300 |
| 618 | 492 |
| 619 | 493 |
| 620 | 387 |
| 621 | 395 |
| 622 | 485 |
| 623 | 486 |
| 624 | 487 |
| 625 | 488 |
| 626 | 489 |
| 627 | 490 |
| 628 | 491 |
| 629 | 494 |
| 630 | 328 |
| 631 | 388 |
| 632 | 389 |
| 633 | 390 |
| 634 | 514 |
| 635 | 515 |
| 636 | 516 |
| 637 | 517 |
| 638 | 518 |
| 639 | 519 |
| 640 | 520 |
| 641 | 522 |
| 642 | 523 |
| 643 | 524 |
| 644 | 525 |
| 645 | 526 |
| 646 | 527 |
| 647 | 528 |
| 648 | 529 |
| 649 | 530 |
| 650 | 531 |
| 651 | 532 |
| 652 | 533 |
| 653 | 534 |
| 654 | 535 |
| 655 | 536 |
| 656 | 537 |
| 657 | 538 |
| 658 | 539 |
| 659 | 540 |
| 660 | 541 |
| 661 | 542 |
| 662 | 543 |
| 663 | 544 |
| 664 | 545 |
| 665 | 546 |
| 666 | 547 |
| 667 | 548 |
| 668 | 550 |
| 669 | 551 |
| 670 | 552 |
| 671 | 553 |
| 672 | 554 |
| 673 | 555 |
| 674 | 556 |
| 675 | 557 |
| 676 | 558 |
| 677 | 559 |
| 678 | 560 |
| 679 | 561 |
| 680 | 562 |
| 681 | 563 |
| 682 | 564 |
| 683 | 565 |
| 684 | 566 |
| 685 | 567 |
| 686 | 568 |
| 687 | 569 |
| 688 | 570 |
| 689 | 571 |
| 690 | 572 |
| 691 | 573 |
| 692 | 574 |
| 693 | 575 |
| 694 | 576 |
| 695 | 577 |
| 696 | 578 |
| 697 | 579 |
| 698 | 580 |
| 699 | 581 |
| 700 | 582 |
| 701 | 583 |
| 702 | 584 |
| 703 | 585 |
| 704 | 586 |
| 705 | 587 |
| 706 | 588 |
| 707 | 589 |
| 708 | 590 |
| 709 | 591 |
| 710 | 592 |
| 711 | 593 |
| 712 | 594 |
| 713 | 595 |
| 714 | 596 |
| 715 | 597 |
| 716 | 598 |
| 717 | 599 |
| 718 | 600 |
| 719 | 601 |
| 720 | 602 |
| 721 | 603 |
| 722 | 604 |
| 723 | 605 |
| 724 | 606 |
| 725 | 607 |
| 726 | 608 |
| 727 | 609 |
| 728 | 610 |
| 729 | 611 |
| 730 | 612 |
| 731 | 613 |
| 732 | 614 |
| 733 | 615 |
| 734 | 616 |
| 735 | 617 |
| 736 | 618 |
| 737 | 619 |
| 738 | 620 |
| 739 | 621 |
| 740 | 622 |
| 741 | 623 |
| 742 | 624 |
| 743 | 625 |
| 744 | 626 |
| 745 | 627 |
| 746 | 628 |
| 747 | 629 |
| 748 | 630 |
| 749 | 631 |
| 750 | 632 |
| 751 | 633 |
| 752 | 634 |
| 753 | 635 |
| 754 | 636 |
| 755 | 637 |
| 756 | 638 |
| 757 | 639 |
| 758 | 640 |
| 759 | 641 |
| 760 | 642 |
| 761 | 643 |
| 762 | 644 |
| 763 | 645 |
| 764 | 646 |
| 765 | 647 |
| 766 | 648 |
| 767 | 649 |
| 768 | 650 |
| 769 | 651 |
| 770 | 652 |
| 771 | 653 |
| 772 | 654 |
| 773 | 655 |
| 774 | 656 |
| 775 | 657 |
| 776 | 658 |
| 777 | 659 |
| 778 | 660 |
| 779 | 661 |
| 780 | 662 |
| 781 | 663 |
| 782 | 664 |
| 783 | 665 |
| 784 | 666 |
| 785 | 667 |
| 786 | 668 |
| 787 | 669 |
| 788 | 671 |
| 789 | 672 |
| 790 | 673 |
| 791 | 674 |
| 792 | 675 |
| 793 | 676 |
| 794 | 677 |
| 795 | 678 |
| 796 | 679 |
| 797 | 680 |
| 798 | 681 |
| 799 | 682 |
| 800 | 683 |
| 801 | 684 |
| 802 | 685 |
| 803 | 686 |
| 804 | 687 |
| 805 | 688 |
| 806 | 689 |
| 807 | 690 |
| 808 | 691 |
| 809 | 692 |
| 810 | 693 |
| 811 | 694 |
| 812 | 701 |
| 813 | 702 |
| 814 | 703 |
| 815 | 704 |
| 816 | 705 |
| 817 | 706 |
| 818 | 707 |
| 819 | 708 |
| 820 | 709 |
| 821 | 710 |
| 822 | 711 |
| 823 | 712 |
| 824 | 713 |
| 825 | 714 |
| 826 | 715 |
| 827 | 716 |
| 828 | 717 |
| 829 | 718 |
| 830 | 719 |
| 831 | 720 |
| 832 | 721 |
| 833 | 722 |
| 834 | 723 |
| 835 | 724 |
| 836 | 725 |
| 837 | 726 |
| 838 | 727 |
| 839 | 728 |
| 840 | 729 |
| 841 | 730 |
| 842 | 731 |
| 843 | 732 |
| 844 | 733 |
| 845 | 734 |
| 846 | 735 |
| 847 | 736 |
| 848 | 737 |
| 849 | 738 |
| 850 | 739 |
| 851 | 740 |
| 852 | 741 |
| 853 | 742 |
| 854 | 743 |
| 855 | 744 |
| 856 | 745 |
| 857 | 746 |
| 858 | 747 |
| 859 | 748 |
| 860 | 749 |
| 861 | 750 |
| 862 | 751 |
| 863 | 752 |
| 864 | 753 |
| 865 | 754 |
| 866 | 755 |
| 867 | 756 |
| 868 | 757 |
| 869 | 758 |
| 870 | 759 |
| 871 | 760 |
| 872 | 761 |
| 873 | 762 |
| 874 | 763 |
| 875 | 764 |
| 876 | 765 |
| 877 | 766 |
| 878 | 767 |
| 879 | 768 |
| 880 | 769 |
| 881 | 770 |
| 882 | 777 |
| 883 | 778 |
| 884 | 779 |
| 885 | 780 |
| 886 | 781 |
| 887 | 782 |
| 888 | 783 |
| 889 | 784 |
| 890 | 785 |
| 891 | 786 |
| 892 | 787 |
| 893 | 788 |
| 894 | 789 |
| 895 | 790 |
| 896 | 791 |
| 897 | 793 |
| 898 | 794 |
| 899 | 795 |
| 900 | 796 |
| 901 | 797 |
| 902 | 798 |
| 903 | 799 |
| 904 | 800 |
| 905 | 801 |
| 906 | 802 |
| 907 | 803 |
| 908 | 804 |
| 909 | 805 |
| 910 | 806 |
| 911 | 807 |
| 912 | 808 |
| 913 | 809 |
| 914 | 810 |
| 915 | 811 |
| 916 | 812 |
| 917 | 813 |
| 918 | 814 |
| 919 | 815 |
| 920 | 816 |
| 921 | 817 |
| 922 | 818 |
| 923 | 819 |
| 924 | 820 |
| 925 | 821 |
| 926 | 822 |
| 927 | 823 |
| 928 | 824 |
| 929 | 825 |
| 930 | 826 |
| 931 | 827 |
| 932 | 828 |
| 933 | 829 |
| 934 | 830 |
| 935 | 831 |
| 936 | 832 |
| 937 | 833 |
| 938 | 834 |
| 939 | 835 |
| 940 | 836 |
| 941 | 837 |
| 942 | 838 |
| 943 | 839 |
| 944 | 840 |

In some embodiments, a glycoprotein particle of the disclosure comprises a payload comprising a CasX protein of SEQ ID NOS: 574-944 as set forth in Table 2, or a sequence with at least 70%, at least 80%, at least 90%, at least 95% identity thereto. In some embodiments, a glycoprotein particle of the disclosure comprises a payload comprising a CasX protein of SEQ ID NOS: 574-944 as set forth in Table 2. In some embodiments, a glycoprotein particle of the disclosure comprises a payload comprising a nucleic acid encoding a CasX protein of SEQ ID NOS: 574-944 as set forth in Table 2, or a sequence with at least 70%, at least 80%, at least 90%, at least 95% identity thereto. In some embodiments, a glycoprotein particle of the disclosure comprises a payload comprising a nucleic acid encoding a CasX protein of SEQ ID NOS: 574-944 as set forth in Table 2.

TABLE 3
Variant guide RNA sequences

| SEQ ID NO: | Variant |
|---|---|
| 945 | 174 |
| 946 | 175 |
| 947 | 176 |
| 948 | 177 |
| 949 | 179 |
| 950 | 181 |
| 951 | 182 |
| 952 | 183 |
| 953 | 184 |
| 954 | 185 |
| 955 | 186 |
| 956 | 187 |
| 957 | 188 |
| 958 | 189 |
| 959 | 190 |
| 960 | 191 |
| 961 | 192 |
| 962 | 193 |
| 963 | 195 |
| 964 | 196 |
| 965 | 197 |
| 966 | 198 |
| 967 | 199 |
| 968 | 200 |
| 969 | 201 |
| 970 | 202 |
| 971 | 203 |
| 972 | 204 |
| 973 | 205 |
| 974 | 206 |
| 975 | 207 |
| 976 | 208 |
| 977 | 209 |
| 978 | 210 |
| 979 | 211 |
| 980 | 212 |
| 981 | 213 |
| 982 | 214 |
| 983 | 215 |
| 984 | 216 |
| 985 | 217 |
| 986 | 218 |
| 987 | 219 |
| 988 | 220 |
| 989 | 221 |
| 990 | 222 |
| 991 | 223 |
| 992 | 224 |
| 993 | 225 |
| 994 | 226 |
| 995 | 227 |
| 996 | 228 |
| 997 | 229 |
| 998 | 230 |
| 999 | 231 |
| 1000 | 232 |
| 1001 | 233 |
| 1002 | 234 |
| 1003 | 235 |
| 1004 | 236 |
| 1005 | 237 |
| 1006 | 238 |
| 1007 | 239 |
| 1008 | 240 |
| 1009 | 241 |
| 1010 | 242 |
| 1011 | 243 |
| 1012 | 244 |
| 1013 | 245 |
| 1014 | 246 |
| 1015 | 247 |
| 1016 | 248 |
| 1017 | 249 |
| 1018 | 250 |
| 1019 | 251 |
| 1020 | 252 |
| 1021 | 253 |
| 1022 | 254 |
| 1023 | 255 |
| 1024 | 256 |
| 1025 | 257 |
| 1026 | 258 |
| 1027 | 259 |
| 1028 | 260 |
| 1029 | 261 |
| 1030 | 262 |
| 1031 | 263 |
| 1032 | 264 |
| 1033 | 265 |
| 1034 | 266 |
| 1035 | 267 |
| 1036 | 268 |
| 1037 | 269 |
| 1038 | 270 |
| 1039 | 271 |
| 1040 | 272 |
| 1041 | 273 |
| 1042 | 274 |
| 1043 | 275 |
| 1044 | 276 |
| 1045 | 277 |
| 1046 | 278 |
| 1047 | 279 |
| 1048 | 280 |
| 1049 | 281 |
| 1050 | 282 |
| 1051 | 283 |
| 1052 | 284 |
| 1053 | 285 |
| 1054 | 286 |
| 1055 | 287 |
| 1056 | 288 |
| 1057 | 289 |
| 1058 | 290 |
| 1059 | 291 |
| 1060 | 292 |
| 1061 | 293 |
| 1062 | 294 |
| 1063 | 295 |
| 1064 | 296 |
| 1065 | 297 |
| 1066 | 298 |
| 1067 | 299 |
| 1068 | 300 |
| 1069 | 301 |
| 1070 | 302 |
| 1071 | 303 |
| 1072 | 304 |
| 1073 | 305 |
| 1074 | 306 |
| 1075 | 307 |
| 1076 | 308 |
| 1077 | 309 |
| 1078 | 310 |
| 1079 | 311 |
| 1080 | 312 |
| 1081 | 313 |
| 1082 | 314 |
| 1083 | 315 |
| 1084 | 317 |
| 1085 | 318 |
| 1086 | 319 |
| 1087 | 320 |
| 1088 | 321 |
| 1089 | 322 |
| 1090 | 323 |
| 1091 | 324 |
| 1092 | 325 |
| 1093 | 326 |
| 1094 | 327 |
| 1095 | 328 |
| 1096 | 329 |
| 1097 | 330 |
| 1098 | 331 |
| 1099 | 332 |
| 1100 | 333 |
| 1101 | 334 |
| 1102 | 335 |
| 1103 | 336 |
| 1104 | 337 |
| 1105 | 338 |
| 1106 | 339 |
| 1107 | 340 |
| 1108 | 341 |
| 1109 | 343 |
| 1110 | 344 |
| 1111 | 345 |
| 1112 | 346 |
| 1113 | 347 |
| 1114 | 348 |
| 1115 | 349 |
| 1116 | 350 |
| 1117 | 351 |
| 1118 | 352 |
| 1119 | 353 |
| 1120 | 354 |
| 1121 | 355 |
| 1122 | 356 |
| 1123 | 357 |
| 1124 | 358 |
| 1125 | 359 |
| 1126 | 360 |
| 1127 | 361 |
| 1128 | 362 |
| 1129 | 363 |
| 1130 | 364 |
| 1131 | 365 |
| 1132 | 366 |
| 1133 | 367 |
| 1134 | 368 |
| 1135 | 369 |
| 1136 | 370 |
| 1137 | 371 |
| 1138 | 372 |
| 1139 | 373 |
| 1140 | 374 |
| 1141 | 375 |

In some embodiments, a glycoprotein particle of the disclosure comprises a payload comprising a guide RNA comprising a scaffold of SEQ ID NOS: 945-1141 as set forth in Table 3, or a sequence with at least 70%, at least 80%, at least 90%, at least 95% identity thereto. In some embodiments, a glycoprotein particle of the disclosure comprises a payload comprising a guide RNA comprising a scaffold of SEQ ID NOS: 945-1141 as set forth in Table 3.

In some embodiments, a glycoprotein particle of the disclosure comprises a payload comprising a CasX protein of SEQ ID NOS: 574-944 as set forth in Table 2, or a sequence with at least 70%, at least 80%, at least 90%, at least 95% identity thereto, and a guide RNA variant of SEQ ID NOS: 945-1141 as set forth in Table 3, or a sequence with at least 70%, at least 80%, at least 90%, at least 95% identity thereto. In some embodiments, a glycoprotein particle of the disclosure comprises a payload comprising a CasX protein of SEQ ID NOS: 574-944 as set forth in Table 2, and a guide RNA comprising a scaffold of SEQ ID NOS: 945-1141 as set forth in Table 3. In some embodiments, the CasX and the gRNA are associated as a ribonucleoprotein. In some embodiments, the gRNA comprises a targeting sequence having 15 to 30 nucleotides that is complementary to, and therefore hybridizes with, a target nucleic acid in a cell, and is linked to the 3′ end of the gRNA scaffold sequence.

Glycoprotein particles comprising additional gene editing systems, or nucleic acids encoding additional gene editing systems, are also envisaged as within the scope of the instant disclosure. In some embodiments, the gene editing system comprises a zinc finger nuclease (ZFN). Custom-designed ZFNs that combine the non-specific cleavage domain, for example a FokI endonuclease domain, with zinc finger domains that bind to specific target sequences can create site-specific double-strand breaks in a target nucleic acid. In some embodiments, the gene editing system comprises a transcription activator like effector nuclease (TALEN). Transcription Activator-Like Effector Nucleases (TALENs) are artificial restriction enzymes generated by fusing the TAL effector DNA binding domain to a DNA cleavage domain such as FokI. These reagents enable efficient, programmable, and specific DNA cleavage and represent powerful tools for genome editing in situ. Transcription activator-like effectors (TALEs) can be quickly engineered to bind practically any DNA sequence.

In some embodiments, a glycoprotein particle of the disclosure comprises a small molecule for a therapeutic or diagnostic application. In some embodiments, the glycoprotein particle encapsulates the small molecule. In some embodiments, the glycoprotein particle is covalently or non-covalently attached to the small molecule. In some embodiments, the small molecule is under 1 KD in weight. In some embodiments, the glycoprotein particle confers enhanced tissue specific tropism to the small molecule, and/or lowers the dosage required of the small molecule to treat or diagnose a subject in need thereof.

### V. Methods of Use

Glycoprotein particles of the disclosure may be used for any application in which cell or tissue targeting is desired. In some embodiments, glycoprotein particles of the disclosure are used in therapeutic applications. In other embodiments, glycoprotein particles of the disclosure are used in diagnostic applications, such as imaging.

In some embodiments, a glycoprotein particle of the disclosure comprises a therapeutic payload and is administered to treat a disease or disorder in a subject in need thereof. In some embodiments, the disease or disorder is selected from the group consisting of cancers, immunoregulatory diseases, pulmonary diseases and disorders, cardiovascular diseases, infectious diseases, genetic disorders, neurological disorders, endocrine disorders, metabolic disorders, intestinal diseases or disorders, mental illnesses, sexually transmitted diseases, gynecological diseases, urogenital diseases, skin diseases, and ocular diseases.

In some embodiments, the disease is cancer. In some embodiments, the cancer comprises a solid tumor or a liquid tumor. Exemplary cancers comprising solid tumors, include, but are not limited to, breast cancer, lung cancer, prostate cancer and skin cancer. Exemplary cancers comprising liquid tumors include lymphomas and leukemias.

In some embodiments, a glycoprotein particle of the disclosure comprises any of the therapeutic payloads described herein, e.g., a gene editing pair comprising a CasX protein of Table 2 and a gRNA comprising a scaffold of Table 3, for use in treating a disease in a subject in need thereof, wherein the glycoprotein particle delivers the therapeutic payload to the diseased cell or tissue.

In some embodiments, delivery of a therapeutic payload treats the disease or disorder in the subject. In some embodiments, delivery of a therapeutic payload reduces a sign or a symptom of the disease or disorder in the subject. In some embodiments, the therapeutic payload is administered as part of an ongoing treatment.

In some embodiments, a glycoprotein particle of the disclosure comprises a diagnostic payload to diagnose a subject at risk for developing a disease or disorder, or suspected of having a disease or disorder. In some embodiments, a glycoprotein particle comprises an imaging moiety, e.g., a fluorophore or inorganic metal for use in diagnostic imaging, wherein the glycoprotein protein targets and delivers the imaging moiety to the cell or tissue of interest. Exemplary fluorophores include both proteins such as green fluorescent protein (GFP), red fluorescent protein (RFP) and their derivatives, as well as inorganic fluorophores, such as dyes. Exemplary fluorescent dyes include but are not limited to, 7-Amino-actinomycin D, Acridine orange, Acridine yellow, Alexa Fluor dyes (Molecular Probes), Auramine O, Auramine-rhodamine stain, Benzanthrone, 9,10-Bis(phenylethynyl)anthracene, 5,12-Bis(phenylethynyl)naphthacene, CFDA-SE, CFSE, Calcein, Carboxyfluorescein, 1-Chloro-9,10-bis(phenylethynyl)anthracene, 2-Chloro-9,10-bis(phenylethynyl)anthracene, Coumarin, Cyanine, DAPI, Dark quencher, Dioc6, DyLight Fluor dyes (Thermo Fisher Scientific), Ethidium bromide, Fluorescein, Fura-2, Fura-2-acetoxymethyl ester, Green fluorescent protein and derivatives, Hilyte Fluor dyes (AnaSpec), Hoechst stain, Indian yellow, Luciferin, Perylene, Phycobilin, Phycoerythrin, Phycoerythrobilin, Propidium iodide, Pyranine, Rhodamine, RiboGreen, Rubrene, Ruthenium(II) tris(bathophenanthroline disulfonate), SYBR Green, Stilbene, Sulforhodamine 101, TSQ, Texas Red, and Umbelliferone.

In some embodiments, inclusion of a glycoprotein in a glycoprotein particle increases the delivery of the glycoprotein particle to a target cell or tissue, relative to an equivalent particle without the glycoprotein. In some embodiments, inclusion of the glycoprotein in the glycoprotein particle increases the cell or tissue targeting by at least a 2-fold, at least a 3-fold, at least a 4-fold, at least a 5-fold, at least a 10-fold, at least a 15-fold, or at least a 20-fold increase, compared to an equivalent particle without the glycoprotein, when assayed under comparable conditions.

In some embodiments, inclusion of a glycoprotein in a glycoprotein particle comprising a gene editing system increases editing in a target cell or tissue relative to an equivalent particle without the glycoprotein. In some embodiments, inclusion of the glycoprotein increases gene editing by at least 2-fold, at least a 3-fold, at least a 4-fold, at least a 5-fold, at least a 10-fold, at least a 15-fold, or at least a 20-fold relative to a particle without the glycoprotein, when assayed under comparable conditions.

Representative examples demonstrating enhanced binding and uptake of glycoprotein particles of the disclosure to target cells and demonstrating payload delivery, e.g., enhanced gene editing of target nucleic acids, are provided in the Examples.

In some embodiments, a glycoprotein particle as provided herein is formulated as a pharmaceutical composition comprising a pharmaceutically acceptable carrier, diluent or excipient.

### VI. Methods of Making

Also provided herein are methods of making the glycoprotein particles described herein. Glycoprotein particles of the disclosure may comprise viral-like particles, lipid nanoparticles, liposomes, exosomes, organic polymer-based particles, inorganic nanoparticles, or any combination thereof, and are designed to deliver one or more payloads to a target cell or tissue. Glycoproteins can be incorporated into the particles described herein using any methods known in the art, including, but not limited to, incorporation into the membrane by a host cell producing the particle, covalent attachment to one or more components of the particle, and non-covalent attachment to one or more components of the particle.

A glycoprotein particle comprising a viral-like particle (VLP) of the disclosure comprises one or more viral structural proteins that may be assembled in vivo through recombinant expression of viral proteins or in vitro using previously purified viral proteins. In preferred embodiments, a glycoprotein particle comprising a VLP comprises lentiviral structural proteins and is assembled in vivo. In some preferred embodiments, a glycoprotein particle comprising a VLP comprises a lipid membrane derived from the host cell. In some embodiments, a glycoprotein particle is produced from nucleic acid sequences encoding a glycoprotein, or a fragment thereof, and VLP structural proteins, which are encoded on one or more plasmids that are co-transfected into a host cell, e.g., as shown in FIGS. 3, 10, and 11. In some embodiments the nucleic acid sequences encoding the glycoprotein are codon optimized.

In some embodiments, a glycoprotein-containing VLP is assembled when a glycoprotein, or fragment thereof, is trafficked to and integrates into a nascent viral membrane prior to VLP budding from the host cell. In some embodiments, a glycoprotein particle comprises a therapeutic or diagnostic payload, wherein the payload is co-expressed in the host cell, and wherein the therapeutic payload is encapsulated in the VLP during or prior to budding from the host cell. In some embodiments, an encapsulated payload of a glycoprotein VLP comprises a genetic editing system comprising a CasX protein and a guide RNA.

In some embodiments, a glycoprotein particle comprises an exosome, derived from a host cell expressing the glycoprotein, or fragment thereof. Exosomes comprising the glycoproteins described herein can be produced by any suitable cell type expressing the glycoprotein, including, but not limited to, mammalian cells such as CHO, HeLa or Jurkat cells. In some embodiments, the glycoprotein particle comprising an exosome comprises a therapeutic payload expressed in the host cell. Exosomes may be isolated by methods known in the art, to isolate exosomes based on their density and size differences from other components in a sample, i.e., size-exclusion chromatography, precipitation, affinity capture or various centrifugation techniques.

In some embodiments, a glycoprotein particle comprises a liposome or lipid nanoparticle (LNP), wherein the lipid particle is assembled in vitro. A glycoprotein particle comprising a lipid particle may be assembled, for example, in a microfluidics system wherein the membrane bound glycoprotein, or fragment thereof, is mixed in the lipid phase with the other lipid components. In some embodiments, a glycoprotein particle comprising a lipid particle comprises a payload. In some embodiments, the payload is incorporated into the liposome or LNP in an aqueous phase, wherein the payload and the lipid components, i.e., a glycoprotein and LNP components, are mixed using a controlled flow rate.

In some embodiments, a glycoprotein particle comprises an organic polymer-based particle, e.g., a particle comprising one or more of polyacrylates, polyalkylcyanoacrylates, polylactide, polylactide-polyglycolide copolymers, polycaprolactones, dextran, albumin, gelatin, alginate, collagen, chitosan, cyclodextrins, protamine, polyethylene glycol (PEG)-modified (PEGylated) protamine, poly-D-lysine (PLL), PEGylated PLL, polyethylenimine (PEI), or poly (lactic-co-glycolic acid) (PLGA).

Organic polymers may be made according to methods known in the art, and are commercially available from chemical suppliers such as Sigma Aldrich. In some embodiments, a glycoprotein particle comprises a particle comprising poly (lactic-co-glycolic acid) (PLGA). PLGA particles may be made, for example, using the emulsification-solvent evaporation and nanoprecipitation techniques as described in Hernández-Giottonini et al. Royal Society of Chemistry Advances. 2020. v10:4218-4231.

In some embodiments, a glycoprotein particle comprises an inorganic particle, e.g., comprising gold, iron, calcium phosphate and/or silica that is conjugated to a glycoprotein by methods known in the art. In some embodiments, a glycoprotein may comprise for example, a gold nanoparticle which allows for cysteine-gold covalent bonding, and/or electrostatic attachment of the nanoparticle to charged groups of the protein.

In some embodiments, the glycoprotein, or fragment thereof, is conjugated to the surface of a particle, e.g., to a viral-like particle (VLP), a liposome, an exosome, a lipid nanoparticle (LNP), an organic polymer-based particle, and/or an inorganic particle. In some embodiments, the glycoprotein is a biotinylated glycoprotein, and one or more components of the particle (other than the glycoprotein) comprise an avidin or streptavidin component. The biotinylated glycoprotein then binds to the avidin or streptavidin component of the particle, thereby producing a glycoprotein particle wherein the glycoprotein is conjugated to the particle via the biotin/avidin or biotin/streptavidin interaction.

In some embodiments, a glycoprotein particle comprises one or more components of the particle (other than the glycoprotein) that have been modified to have a chemically reactive functional groups for conjugation of the glycoprotein to the particle. For example, PLGA polymers for use in making a glycoprotein particle can be modified to comprise a COOH group to bind amino acids through the conventional carbodiimide coupling reaction. Alternatively, or in addition, the PLGA polymers can be modified to include a maleimide group to bind thiol-containing amino acids. In some embodiments, an inorganic particle, e.g., a gold nanoparticle, is functionalized with PEG thiols to facilitate attachment of a properly folded glycoprotein, as described in Aubin-Tam, Methods of Molecular Biology 2013:1025:19-27.

In some embodiments, the glycoprotein, or fragment thereof comprises a modified or non-natural amino acid functionalized for conjugation of the glycoprotein with the particle. For example, the glycoprotein, or fragment thereof, can incorporate an amino acid functionalized to bind a particle using an alkyne/azide click reaction, carbonyl condensation, Michael-type addition, or Mizoroki-Heck substitution, all of which are known to persons of skill in the art.

The payload can be incorporated or attached to the glycoprotein particle using any methods known in the art. In some embodiments, such as the VLP, LNP, liposomes and organic polymer-based particles described supra, the payload is incorporated within the particle, e.g., within a lumen of the particle. Alternatively, or in addition, the payload may be incorporated into the membrane or shell of the particle, or attached to the particle using covalent or non-covalent attachment.

In some embodiments, the glycoprotein particle comprises a payload conjugated to the glycoprotein particle. In some embodiments, for example, a glycoprotein particle comprises an inorganic particle conjugated to a fluorophore or other imaging moiety for diagnostic use.

### ENUMERATED EMBODIMENTS

The invention may be defined by reference to the following sets of enumerated, illustrative embodiments:

### Set I:

Embodiment I-1. A particle comprising a glycoprotein as set forth in any one of the sequences of Table 1, a sequence comprising at least 70% identity thereto (variant), or a fragment thereof.

Embodiment I-2. The particle of embodiment I-1, wherein the particle is a microparticle.

Embodiment I-3. The particle of embodiment I-1, wherein the particle is a nanoparticle.

Embodiment I-4. The particle of embodiment I-1, wherein the particle comprises one or more of fullerenes, metals, biological polymers, dendrimers, quantum dots, lipids, nucleic acid vectors, and viral structural proteins.

Embodiment I-5. The particle of embodiment I-3, wherein the particle is a polymeric nanoparticle.

Embodiment I-6. The particle of embodiment I-5, wherein the polymeric nanoparticle comprises a nanocapsule or a nanosphere.

Embodiment I-7. The method of embodiment I-5, wherein the polymeric nanoparticle comprises a polymer micelle.

Embodiment I-8. The particle of any one of embodiments I-1 to I-7, wherein the particles are non-toxic and/or biodegradable.

Embodiment I-9. The particle of any one of embodiments I-1 to I-3, wherein the particle is a lipid-based particle.

Embodiment I-10. The particle of embodiment I-9, wherein the lipid-based particle comprises a lipid micelle or a liposome.

Embodiment I-11. The particle of embodiment I-1, wherein the particle is a viral vector.

Embodiment I-12. The particle of embodiment I-1, wherein the particle is a virus-like particle (VLP).

Embodiment I-13. The particle of embodiment I-12, wherein the VLP is derived from a virus selected from the group consisting of adeno-associated virus (AAV), adenovirus, retrovirus and herpes simplex virus.

Embodiment I-14. The particle of embodiment I-13, wherein the retrovirus comprises an Orthoretrovirinae virus or a Spumaretrovirinae virus.

Embodiment I-15. The particle of embodiment I-14, wherein the Orthoretrovirinae virus is selected from the group consisting of an Alpharetrovirus, Betaretrovirus, Deltaretrovirus, Epsilonretrovirus, Gammaretrovirus, and Lentivirus.

Embodiment I-16. The particle of embodiment I-14, wherein the Spumaretrovirinae virus is selected from the group consisting of Bovispumavirus, Equispumavirus, Felispumavirus, Prosimiispumavirus, Simiispumavirus, or Spumavirus.

Embodiment I-17. The particle of any one of embodiments I-1 to I-16, wherein the glycoprotein, variant, or fragment thereof is expressed on the surface of the particle.

Embodiment I-18. The particle of embodiment I-17, wherein the glycoprotein enhances targeting of the particle to a cell or tissue.

Embodiment I-19. The particle of embodiment I-18, wherein the targeting is in vitro.

Embodiment I-20. The particle of embodiment I-18, wherein the targeting is in vivo.

Embodiment I-21. The particle of any one of embodiments I-1 to I-20, wherein the particle comprises a payload.

Embodiment I-22. The particle of embodiment I-21, wherein the payload is a protein, nucleic acid, cell, or small molecule.

Embodiment I-23. The particle of embodiment I-21, wherein the payload is a diagnostic.

Embodiment I-24. A pharmaceutical composition comprising any one or more of particles of embodiments I-1 to I-22.

Embodiment I-25. A method of treating a subject in need thereof, comprising administering to the subject the particle of any one of embodiments I-1 to I-22.

### Set II:

Embodiment II-1. A glycoprotein particle comprising a particle and a glycoprotein, wherein the glycoprotein is any one of the sequences of SEQ ID NOS: 1-224, 281-284, or 286-573 as set forth in Table 1, a sequence comprising at least 70%, at least 80%, at least 90%, at least 95%, at least 96%, at least 97%, at least 98%, or at least 99% identity thereto, or a fragment thereof.

Embodiment II-2. The glycoprotein particle of embodiment II-1, wherein the glycoprotein consists of any one of SEQ ID NOS: 1-224, 281-284, or 286-573 as set forth in Table 1.

Embodiment II-3. The glycoprotein particle of embodiment II-1, wherein the glycoprotein fragment is a virion surface domain.

Embodiment II-4. The glycoprotein particle of embodiment II-1, wherein the particle is a microparticle.

Embodiment II-5. The glycoprotein particle of embodiment II-1, wherein the particle is a nanoparticle.

Embodiment II-6. The glycoprotein particle of embodiment II-1, wherein the particle comprises one or more of fullerenes, metals, biological polymers, dendrimers, quantum dots, lipids, nucleic acid vectors, and viral structural proteins.

Embodiment II-7. The glycoprotein particle of embodiment II-5, wherein the particle is a polymeric nanoparticle.

Embodiment II-8. The glycoprotein particle of embodiment II-7, wherein the polymeric nanoparticle comprises a nanocapsule or a nanosphere.

Embodiment II-9. The glycoprotein particle of embodiment II-7, wherein the polymeric nanoparticle comprises a polymer micelle.

Embodiment II-10. The glycoprotein particle of any one of embodiments II-1 to II-9, wherein the particle is non-toxic and/or biodegradable.

Embodiment II-11. The glycoprotein particle of any one of embodiments II-1 to II-5, wherein the particle is a lipid-based particle.

Embodiment II-12. The glycoprotein particle of embodiments II-1 to II-3, wherein the particle is a virus-like particle (VLP).

Embodiment II-13. The glycoprotein particle of embodiment II-12, wherein the VLP is derived from a virus selected from the group consisting of retrovirus, adeno-associated virus (AAV), adenovirus, and herpes simplex virus.

Embodiment II-14. The glycoprotein particle of embodiment II-13, wherein the retrovirus is an Orthoretrovirinae virus or a Spumaretrovirinae virus.

Embodiment II-15. The glycoprotein particle of embodiment II-14, wherein the Orthoretrovirinae virus is selected from the group consisting of an Alpharetrovirus, Betaretrovirus, Deltaretrovirus, Epsilonretrovirus, Gammaretrovirus, and Lentivirus.

Embodiment II-16. The glycoprotein particle of embodiment II-14, wherein the Spumaretrovirinae virus is selected from the group consisting of Bovispumavirus, Equispumavirus, Felispumavirus, Prosimiispumavirus, Simiispumavirus, or Spumavirus.

Embodiment II-17. The glycoprotein particle of embodiments II-1 to II-3, wherein the particle is an exosome.

Embodiment II-18. The glycoprotein particle of any one of embodiments II-1 to II-3, or II-12 to II-17, wherein the glycoprotein is expressed in a host cell and is trafficked to the host cell membrane prior to formation of the glycoprotein particle.

Embodiment II-19. The glycoprotein particle of embodiments II-1 to II-3, wherein the particle comprises a lipid nanoparticle (LNP).

Embodiment II-20. The glycoprotein particle of any one of embodiments II-1 to II-19, wherein the glycoprotein is incorporated into a membrane of the particle.

Embodiment II-21. The glycoprotein particle of any one of embodiments II-1 to II-20, wherein the glycoprotein particle comprises an organic polymer-based particle.

Embodiment II-22. The glycoprotein particle of any one of embodiments II-1 to II-21, wherein the particle comprises an inorganic nanoparticle.

Embodiment II-23. The glycoprotein particle of any one of embodiment II-1 to II-22, wherein the glycoprotein is conjugated to the surface of the particle.

Embodiment II-24. The glycoprotein particle of embodiment II-23, wherein the conjugation comprises covalent attachment or non-covalent attachment.

Embodiment II-25. The glycoprotein particle of embodiments II-1 to II-24, wherein the glycoprotein particle comprises a payload.

Embodiment II-26. The glycoprotein particle of embodiment II-25, wherein inclusion of the glycoprotein in the glycoprotein particles enhances delivery of the payload to a cell or tissue compared to an equivalent particle that does not comprise the glycoprotein.

Embodiment II-27. The glycoprotein particle of embodiments II-24 to II-26, wherein the payload comprises a protein, nucleic acid, small molecule, or a combination thereof.

Embodiment II-28. The glycoprotein particle of embodiments II-25 to II-27, wherein the payload is a therapeutic payload.

Embodiment II-29. The glycoprotein particle of embodiment II-28, wherein delivery of the therapeutic payload to a cell or a tissue of a subject treats a disease or disorder in the subject.

Embodiment II-30. The glycoprotein particle of embodiment II-28 or II-29, wherein the therapeutic payload comprises a protein or an oligonucleotide.

Embodiment II-31. The glycoprotein particle of embodiment II-30, wherein the oligonucleotide comprises a sequence encoding a protein.

Embodiment II-32. The glycoprotein particle of embodiment II-30 or II-31, wherein the protein comprises a cytokine, growth factor, interleukin, enzyme, receptor, microprotein, hormone, RNAse, DNAse, blood clotting factor, anticoagulant, bone morphogenetic protein, engineered protein scaffold, thrombolytics, antibody, antibody fragment, antibody fusion protein, transcription factor, viral interferon antagonist, tick protein, or engineered therapeutic protein.

Embodiment II-33. The glycoprotein particle of embodiment II-30, wherein the oligonucleotide comprises a single-stranded antisense oligonucleotide (ASO), double-stranded RNA interference (RNAi) molecule, DNA aptamer, RNA aptamer, microRNA, ribozyme, RNA decoy, or circular RNA.

Embodiment II-34. The glycoprotein particle of embodiments II-25 to II-27, wherein the payload is a diagnostic payload.

Embodiment II-35. The glycoprotein particle of any one of embodiments II-25 to II-27, wherein the payload comprises a gene editing system.

Embodiment II-36. The glycoprotein particle of embodiment II-35, wherein the gene editing system comprises a Class 2, Type II CRISPR/Cas system, a Class 2, Type V CRISPR/Cas system, a zinc finger nuclease or a TALEN.

Embodiment II-37. The glycoprotein particle of embodiment II-36, wherein the Class 2, Type II CRISPR/Cas system comprises Cas9.

Embodiment II-38. The glycoprotein particle of embodiment II-36, wherein the Class 2, Type V CRISPR/Cas system comprises one or more of CasX, Cas12a (Cpf1), Cas12b (C2c1), Cas12c (C2c3), Cas12d (CasY), Cas12e (CasX), Cas12f, Cas12g, Cas12h, Cas12i, Cas12j, Cas12k, Cas14, and/or CasΦ.

Embodiment II-39. The glycoprotein particle of embodiment II-38, wherein the Class 2, Type V CRISPR/Cas system comprises a gene editing pair comprising a CasX protein comprising an amino acid sequence with at least 90% identity to any one of SEQ ID NOS: 574-944, or as set forth in Table 2, and a guide RNA (gRNA) comprising a scaffold comprising a nucleic acid sequence with at least 90% identity to any one of SEQ ID NOS: 945-1141, or as set forth in Table 3.

Embodiment II-40. The glycoprotein particle of any one of embodiments II-25 to II-30, wherein the payload comprises a gene editing pair comprising a CasX protein of any one of SEQ ID NOS: 574-944, or as set forth in Table 2, and a guide RNA comprising a scaffold of any one of SEQ ID NOS: 945-1141, or as set forth in Table 3.

Embodiment II-41. The glycoprotein particle of embodiment II-39 or II-40, wherein the gRNA further comprises a targeting sequence linked to the 3′ end of the gRNA scaffold sequence.

Embodiment II-42. A pharmaceutical composition comprising a glycoprotein particle of any one of the preceding embodiments and a pharmaceutically acceptable carrier, diluent, or excipient.

Embodiment II-43. A method of treating a subject in need thereof, comprising administering to a subject in need thereof, a glycoprotein particle of any one of embodiments II-1 to II-41 or the pharmaceutical composition of embodiment II-42.

Embodiment II-44. The method of embodiment II-43, wherein the subject has a disease or disorder selected from the group consisting of cancer, an immunoregulatory disease, a pulmonary disease or disorder, a cardiovascular disease, an infectious disease, a genetic disease or disorder, a neurological disease or disorder, an endocrine disease or disorder, a metabolic disease or disorder, an intestinal disease or disorder, a mental illness, a sexually transmitted disease, a gynecological disease, an urogenital disease, a skin disease, or an ocular disease.

Embodiment II-45. The method of embodiment II-43 or II-44, wherein administration of the glycoprotein particle or pharmaceutical composition reduces a sign or a symptom of the disease or disorder.

### EXAMPLES

### Example 1: Construction of a Glycoprotein Particle Comprising a Viral-Like Nanoparticle Delivery System to Evaluate Tropism of Glycoproteins in Mouse Neural Progenitor Cells

Retroviral (e.g., lentiviral) vectors were constructed with an envelope protein of vesicular stomatitis virus (VSV-G), a glycoprotein that endows both a broad host cell range and high vector particle stability. Studies were performed in which viral-like nanoparticle delivery systems incorporated an example payload of a Ribonucleic Protein (RNP) comprising a CasX protein and a guide RNA (gRNA) specific for editing tdTomato in mouse neural progenitor cells (tdT NPCs). These particle delivery systems were created with varying concentrations of VSV-G to determine their effects on delivery to the NPC via the readout of tdTomato expression.

To determine the effects of varying concentrations of the pseudotyping (VSV-G) plasmid, VSV-G was incorporated into the particle delivery system as follows: 1 μg of the VSV-G plasmid was used for the 100% VSV-G group, 0.3 μg was used for the 30% VSV-G group, 0.1 μg was used for the 10% VSV-G group, 0.03 μg was used for the 3% VSV-G group, 0.01 μg was used for the 1% VSV-G group, and 0.003 μg was used for the 0.3% VSV-G group. Titering of the viral particles produced was done using the Takara p24 rapid titer kit. Editing was assessed in the tdTomato NPC cells.

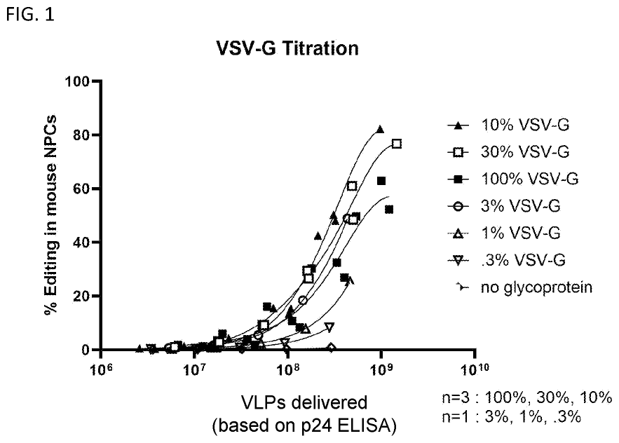

The results for the 10% and 30% VSV-G groups trend towards a better editing outcome as compared to the 100% VSV-G group, as shown in FIG. 1, without affecting viral titer or stability.

As the results indicate, under the experimental conditions, the same or higher editing with 10-30% VSV-G is achieved relative to the 100% VSV-G group. This result opened the possibility of pseudotyping the delivery particle with additional glycoproteins, either with or without VSV-G, to confer differential or enhanced cellular tropism.

Each viral particle transfection used 3.3 μg (0.467 pM) of psPax2 plasmid, 19.8 μg (3.24 pM) of pStx43.119 plasmid, 5 μg (3.13 pM) of pStx42 plasmid targeting the tdTomato locus using spacer 12.7 and 0.262 pM of the respective glycoprotein(s) plasmid which varied in molecular weight. Glycoprotein plasmids contained the same backbone pGP2 and only varied by expressing different viral envelope proteins. Canonical HSV-1 pseudotyping requires four glycoproteins which were used in equimolar amounts in this assay (Polpitiya Arachchige, S., Henke, W., Kalamvoki, M. et al. Analysis of herpes simplex type 1 gB, gD, and gH/gL on production of infectious HIV-1: HSV-1 gD restricts HIV-1 by exclusion of HIV-1 Env from maturing viral particles. Retrovirology 16:9 (2019)). Glycoprotein amino acid sequences come from wild type viral sequences.

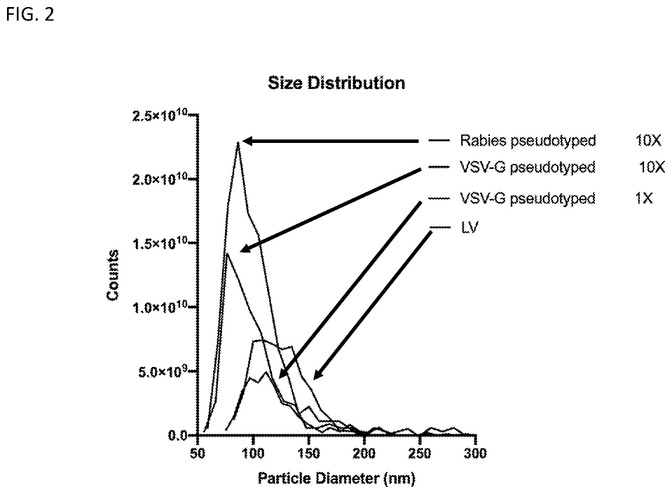

It was also assessed whether pseudotyping with different viral glycoproteins could have an impact on overall size distributions (FIG. 2), which could, in turn, have an impact on in vivo editing efficiencies in different tissues of interest. For this study, the rabies pseudotyped viral particle 10× and VSV-G pseudotyped viral particle 1× were produced using the protocol described above scaled to a 6 well format and using pGP29 in place of the VSV-G pGP2 plasmid. All plasmid quantities and cells used were scaled down 8-fold. The VSV-G pseudotyped viral particle 1× were generated as described above. These preparations were then concentrated at 20,000×g at 4° C. for 90 minutes without a sucrose buffer. Lentivirus was transfected with the following plasmid weights: 5.4 μg of psPax2, 1.8 μg of pGP2, and 7.2 μg of pStx34.119.174.12.7, generating lentivirus designed to induce production and incorporation of RNP with the same enzymatic capabilities as VSV-G pseudotyped viral particle 1×. Samples were diluted appropriately for analysis. The size and number of particles were assessed using a Tunable Resistive Pulse Sensor (Izon Biosciences qNano Gold). While both rabies and VSV-G viral particles ranged in size from 75-140 nm, lentiviruses (LVs) tended to be a bit larger, ranging in size from 85-160 nm, as shown in FIG. 2.

### Example 2: Enhancing Tropism and Editing Potency with Glycoprotein Particles Comprising Vesiculovirus (VSV-G) Glycoprotein Variants

The purpose of these studies was to evaluate the ability of a variety glycoprotein variants to enhance tropism for target cells and improve overall editing of a VLP relative to the standard control VSV-G glycoprotein. VSV-G has been widely used to pseudotype viral vectors. However, VSV-G has been shown to be susceptible to human complement inactivation. Studies were conducted to demonstrate that VSV-G viral particles (with a guide scaffold targeting TdTomato) could be effectively pseudotyped with envelope glycoproteins derived from other species within the Vesiculovirus genus to produce potent particles that successfully edited target cells. This was hypothesized to provide several advantages: 1) some of the variant glycoproteins may be relatively resistant to complement inactivation with human serum; 2) these variant glycoproteins may exhibit enhanced tropism; and 3) viral particles pseudotyped with different glycoproteins may enable repeated dosing of the therapeutic modality, in which different glycoproteins circumvent the humoral immune response induced by the original glycoprotein.

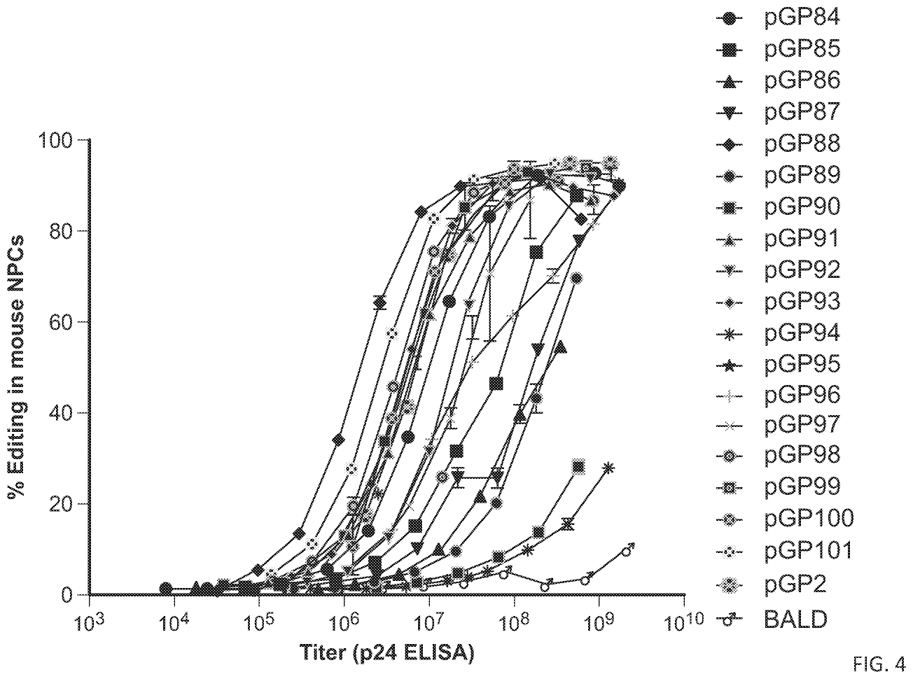

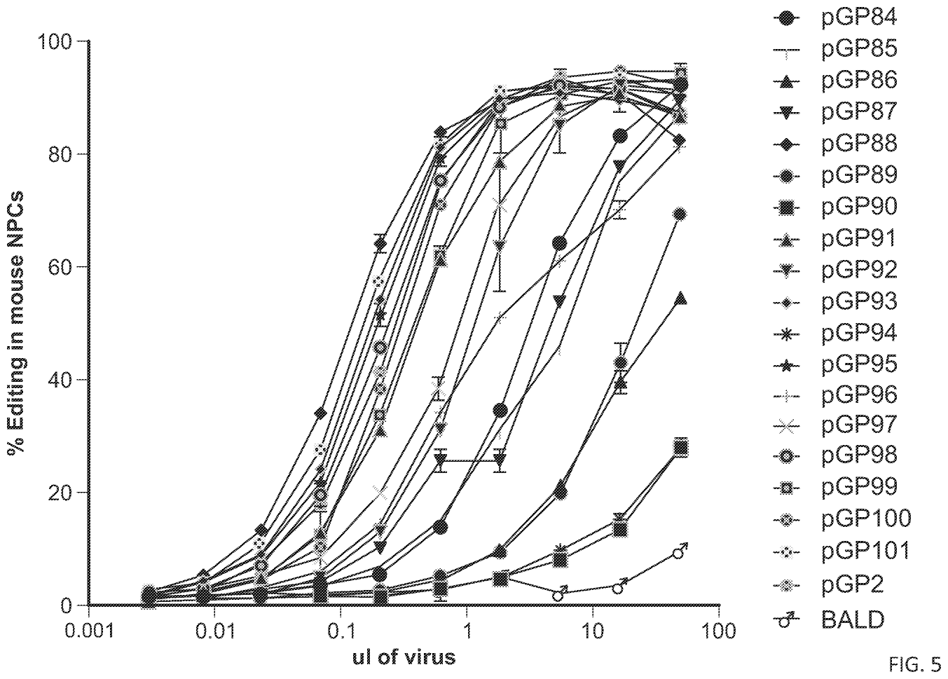

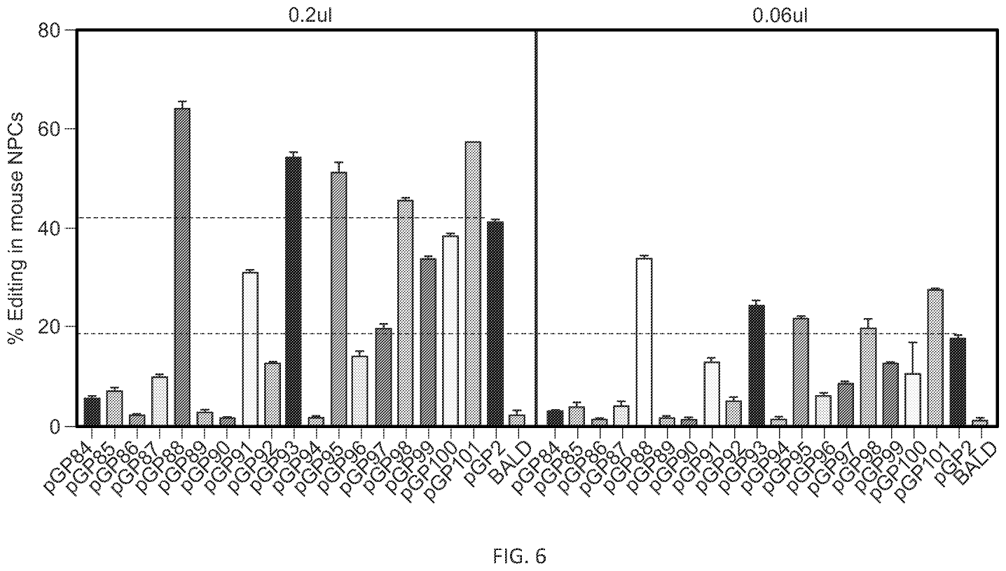

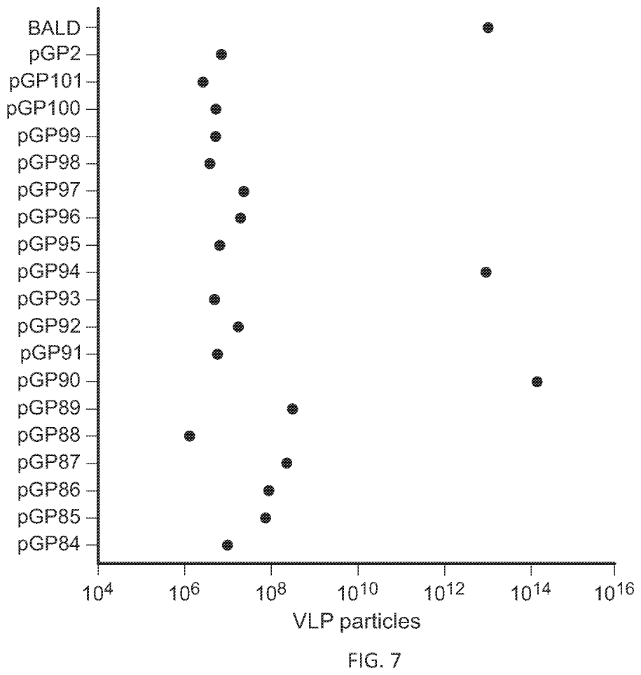

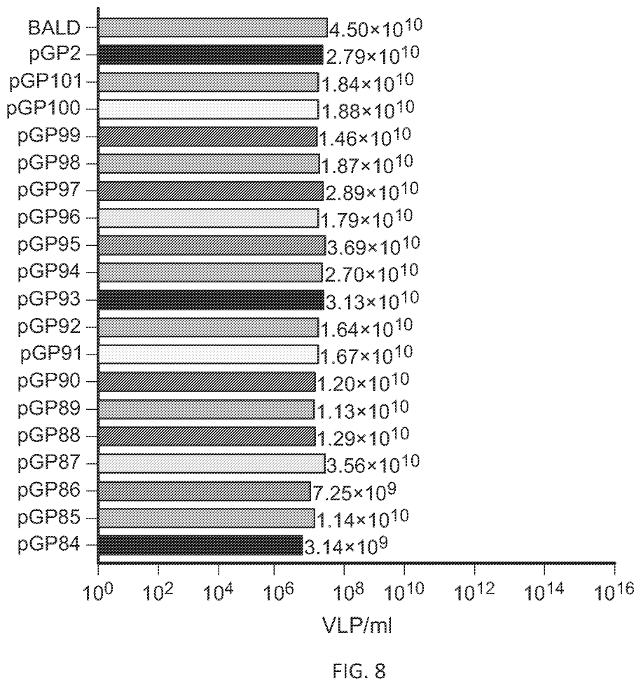

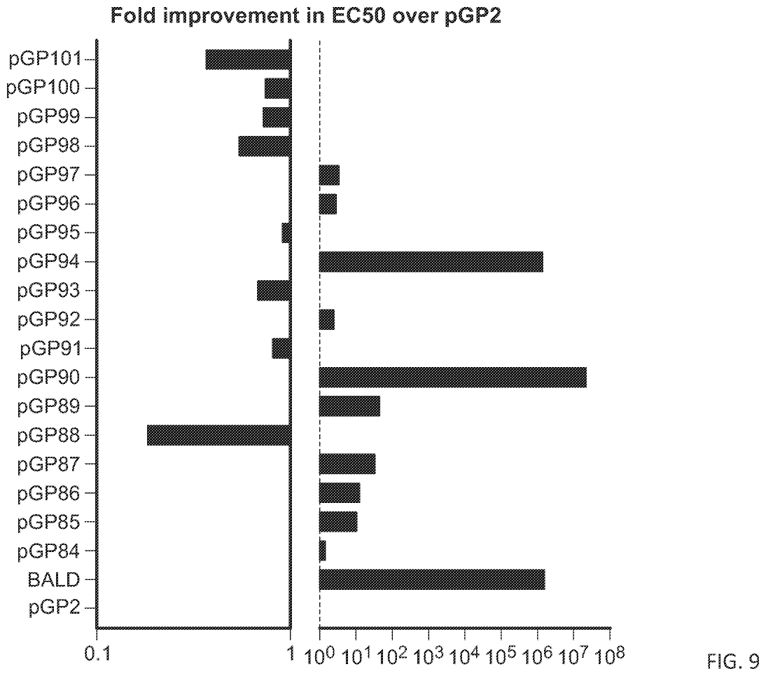

The viral particle was effectively pseudotyped with envelope glycoproteins derived from other species within the Vesiculovirus genus to produce potent particles that successfully edited the target cell (tdT NPCs). In particular, several glycoproteins, including pGP101, pGP100, 99, 98, 95, 93, 91 and 88 (Table 4) showed promise for enhanced tropism and editing capability of the viral particle (FIGS. 4-9).

Example configurations of the glycoprotein sequences within the plasmids are shown in FIGS. 3, 10, and 11.

### Example 3: Enhancing Tropism and Editing Potency with Glycoprotein Particles Comprising Lentiviral and Alphavirus Glycoprotein Variants

The purpose of these studies was to evaluate the ability of diverse glycoprotein variants to enhance the tropism of target cells and improve overall editing of viral particles based on lentiviral and Alphaviral constructs bearing the glycoprotein variants.

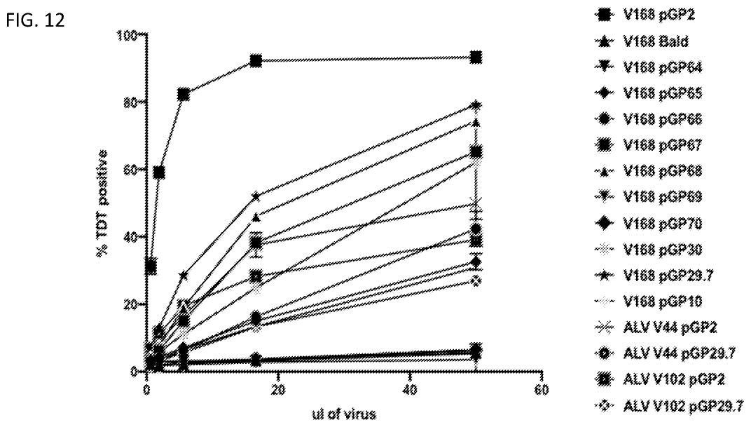

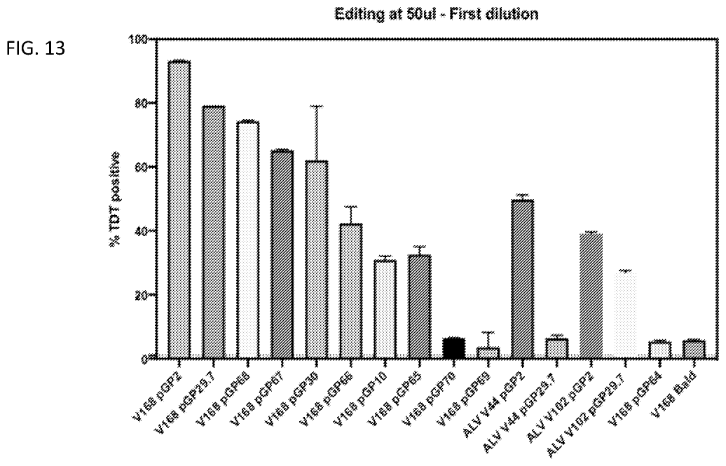

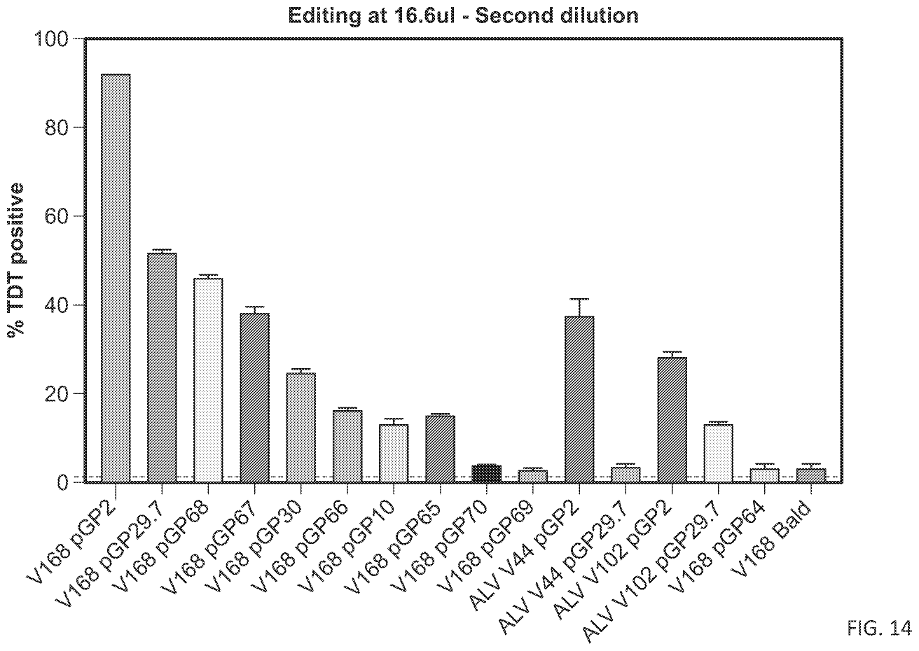

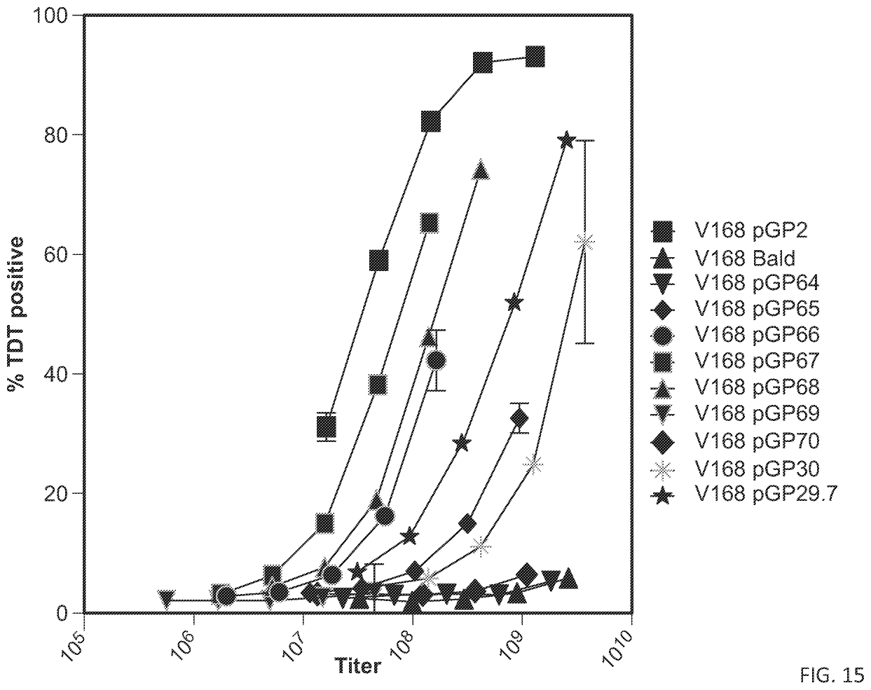

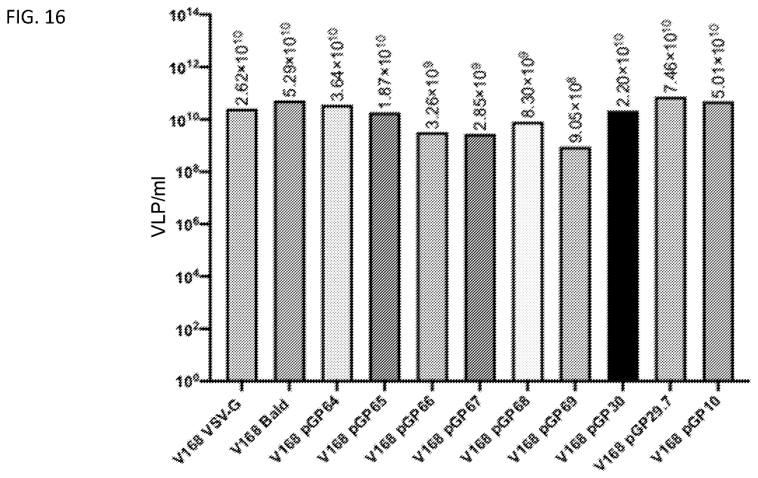

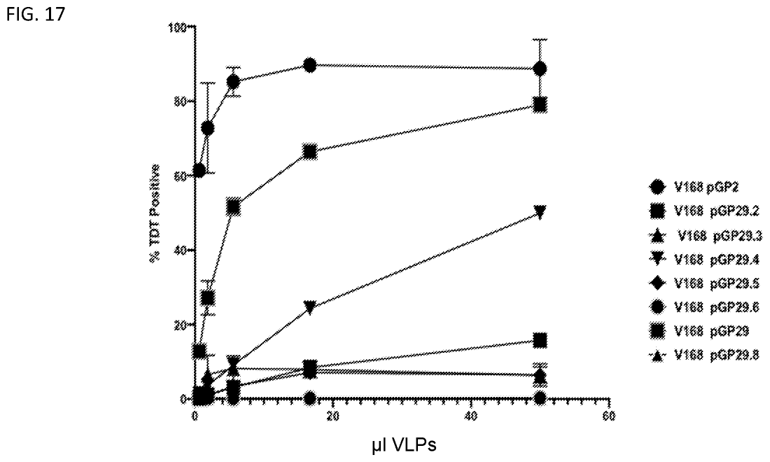

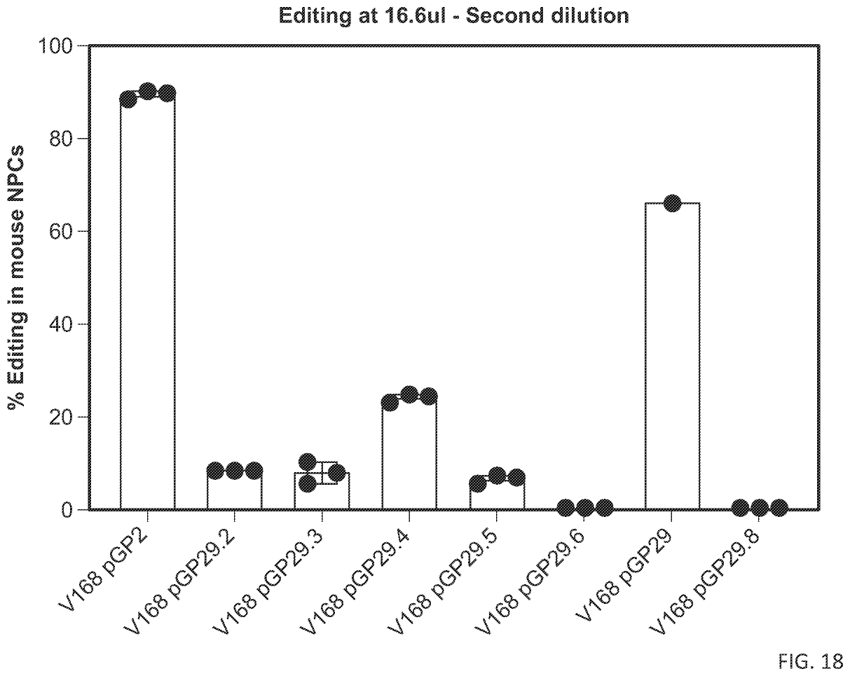

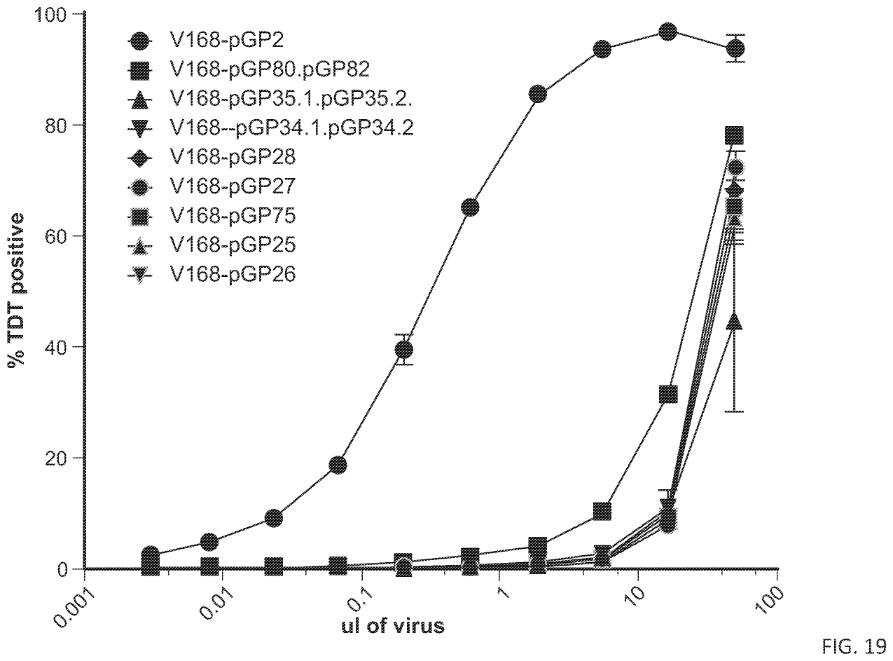

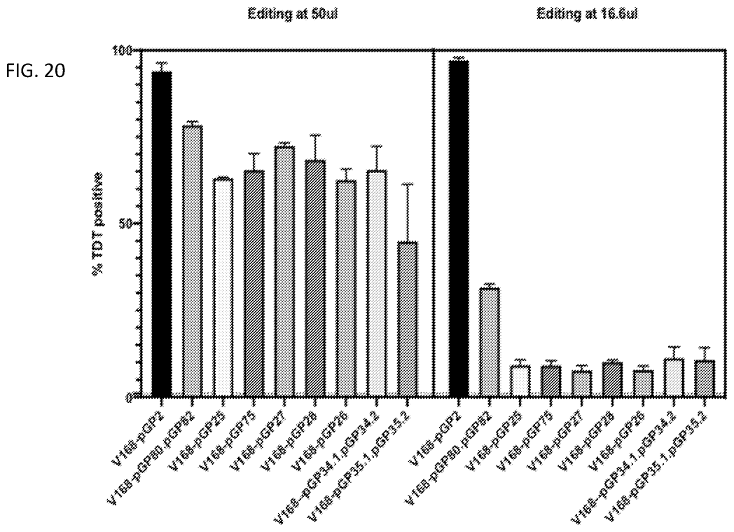

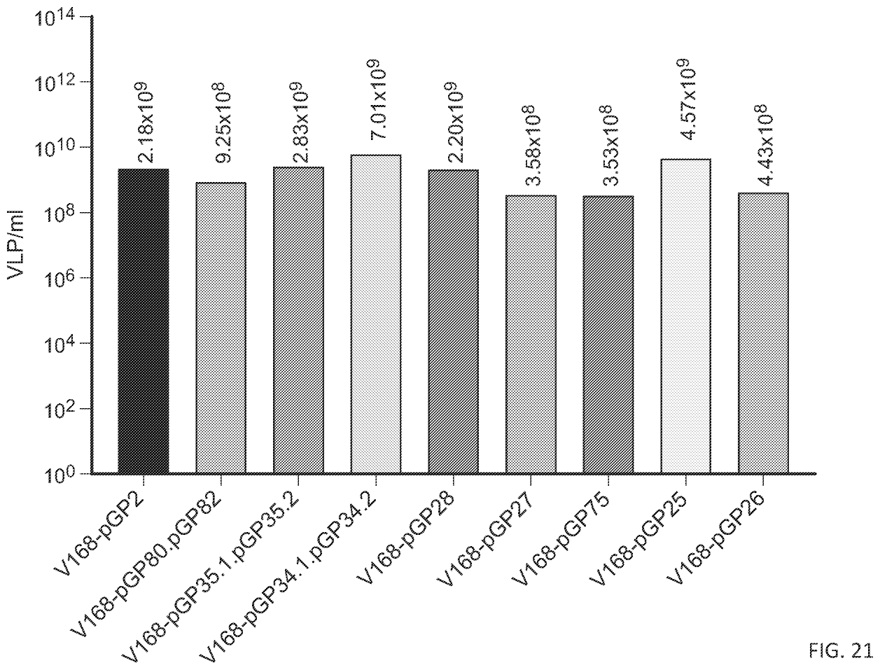

Vesicular stomatitis virus envelope glycoprotein (VSV-G) has been widely used to pseudotyped viral vectors. However, VSV-G has been shown to be susceptible to human complement inactivation. Studies were conducted to demonstrate that viral-like particles derived from lentiviral based HIV (V168 with scaffold 226 targeting TdTomato) as well as other retroviruses such as ALV (V44 and V102 with scaffold 174 targeting TdTomato) were effectively pseudotyped with envelope glycoproteins derived from other viral families including but not limited to Togaviridae, Paramyxoviridae, Rhabdoviridae, Orthomyxoviridae, Retroviridae and Flaviviridae to produce potent particles that successfully edited target cells (FIGS. 12-21). Encoding sequences for these pseudotyped viral-like particles are provided in Table 4. The designed constructs were synthesized as transgenes and purchased pre-cloned into pTWIST expression plasmids from Twist Biosciences. The resultant plasmids were sequenced using Sanger sequencing to ensure correct assembly.

TABLE 4
Plasmid sequences for structural plasmids and glycoproteins

| | viral-like | SEQ |
|---|---|---|
| | Plasmid | ID |
| Version number/Viral source | number | NO |
| H5N1 | pGP80 | 225 |
| H7N9 | pGP81 | 226 |
| Eastern equine encephalitis virus (EEEV) | pGP65 | 227 |
| Venezuelan equine encephalitis viruses (VEEV) | pGP66 | 228 |
| Western equine encephalitis virus (WEEV) | pGP67 | 229 |
| Semliki Forest virus | pGP68 | 230 |
| Sindbis virus | pGP69 | 231 |
| Chikungunya virus (CHIKV) | pGP70 | 232 |
| Bornavirus BoDV-1 | pGP58 | 233 |
| Tick-borne encephalitis virus (TBEV) | pGP71 | 234 |
| Rabies virus (strain Nishigahara RCEH) (RABV) | pGP29.3 | 235 |
| Rabies virus (strain India) (RABV) | pGP29.4 | 236 |
| Rabies virus (strain CVS-11) (RABV) | pGP29.5 | 237 |
| Rabies virus (strain ERA) (RABV) | pGP29.6 | 238 |
| Rabies virus (strain SAD B19) (RABV) | pGP29.7 | 239 |
| Rabies virus (strain Vnukovo-32) (RABV) | pGP29.8 | 240 |
| Rabies virus (strain Pasteur vaccins/PV) (RABV) | pGP29.9 | 241 |
| Rabies virus (strain PM1503/AVO1) (RABV) | pGP29.1 | 242 |
| Rabies virus (strain China/DRV) (RABV) | pGP29.11 | 243 |
| Rabies virus (strain China/MRV) (RABV) | pGP29.12 | 244 |
| Rabies virus (isolate Human/Algeria/1991) (RABV) | pGP29.13 | 245 |
| Rabies virus (strain HEP-Flury) (RABV) | pGP29.14 | 246 |
| Rabies virus (strain silver-haired bat-associated) | pGP29.15 | 247 |
| (RABV) (SHBRV) | | |
| Codon optimized rabies virus | pGP29.2 | 248 |
| Rabies Virus | pGP29 | 249 |
| Mokola Virus | pGP30 | 250 |
| Measles Virus | pGP32.1 | 251 |
| Measles Virus | pGP32.2 | 252 |
| Mouse mammary tumor virus | pGP6 | 253 |
| Human T-lymphotropic virus 1 | pGP7 | 254 |
| RD114 Endogenous Feline Retrovirus | pGP8 | 255 |
| Gibbon ape leukemia virus | pGP9 | 256 |
| Moloney Murine leukemia virus | pGP10 | 257 |
| Baboon Endogenous Virus | pGP11 | 258 |
| Human Foamy Virus | pGP12 | 259 |
| Ebola Zaire Virus | pGP41 | 260 |
| Dengue | pGP25 | 261 |
| Zika virus | pGP26 | 262 |
| West Nile Virus | pGP27 | 263 |
| Japanese Encephalitis Virus | pGP28 | 264 |
| Mumps Virus F | pGP31.1 | 265 |
| Mumps Virus HN | pGP31.2 | 266 |
| Sendai Virus F | pGP33.1 | 267 |
| Sendai Virus HN | pGP33.2 | 268 |
| AcMNPV gp64 | pGP59 | 269 |
| Ross River Virus | pGP54 | 270 |
| N1 Neuraminidase | pGP82 | 271 |
| Dengue virus 2 | pGP75 | 272 |
| Dengue virus 3 | pGP76 | 273 |
| Dengue virus 4 | pGP77 | 274 |
| Nipah Virus | pGP34.1 | 275 |
| Nipah Virus | pGP34.2 | 276 |
| Hendra Virus | pGP35.1 | 277 |
| Hendra Virus | pGP35.2 | 278 |
| Newcastle disease virus | pGP37.1 | 279 |
| Newcastle disease virus | pGP37.2 | 280 |

### Example 4: Screen of Glycoprotein Particles Comprising Pseudotyped Viral Like Particles to Evaluate Tropism of Glycoproteins and Gene Editing Capabilities

The purpose of these experiments was to test whether the cellular tropism of viral-like particles (also referred to herein as XDPs) based on HIV could be altered by pseudotyping the viral-like particles with various viral-derived glycoproteins as targeting moieties.

### Identification of Candidate Glycoproteins

Glycoproteins belonging to a variety of species within the Vesiculovirus, Alphavirus genus, Lyssavirus, and γ-retrovirus genuses were screened for transduction efficiency. The amino acid sequences of the glycoproteins tested are provided in Table 1, above.

Vesiculovirus species glycoprotein investigated herein include Alagoas vesiculovirus, American bat vesiculovirus, Carajas vesiculovirus, Chandipura vesiculovirus, Cocal vesiculovirus, Indiana vesiculovirus, Isfahan vesiculovirus, Jurona vesiculovirus, Malpais Spring vesiculovirus, Maraba vesiculovirus, Morreton vesiculovirus, New Jersey vesiculovirus, Perinet vesiculovirus, Piry vesiculovirus, Radi vesiculovirus, Yug Bogdanovac vesiculovirus, Indiana virus, and New Jersey vesiculovirus. Alagoas vesiculoviruses are known to infect cattle, horses, and pigs. Isfahan vesiculovirus, Chandipura vesiculovirus and Piry vesiculovirus have also been reported to infect humans to cause a flu-like illness.

Alphavirus species glycoproteins investigated herein include Eastern equine encephalitis virus (EEEV), Venezuelan equine encephalitis viruses (VEEV), Western equine encephalitis virus (WEEV), Semliki Forest virus, Sindbis virus and Chikungunya virus (CHIKV). Alphavirus are mostly mosquito-borne viruses known to infect humans, non-human primates, equids, birds, amphibians, reptiles, rodents, and pigs.

Lyssavirus species glycoproteins investigated herein include Rabies and Mokola. γ-retrovirus glycoproteins investigated herein include BABV, GALV and RD114.

### Method for the Generation of VLPs Containing CasX Gene Editing Systems (XDPs)

The screen of glycoproteins was conducted using a five-plasmid system for generating XDPs, using the configurations outlined in Table 5, below.

TABLE 5
Plasmid configurations for generating XDPs

| Plasmid | Encoded Components** |
|---|---|
| 1 | MA*-CA*-NC*-p1/p6-MS2 protein |
| 2 | MA*-CA*-NC*-p1/p6*-Pro† |
| 3 | CasX 676 |
| 4 | Glycoprotein |
| 5 | Single guide RNA, with MS2 tag |

*indicates cleavage sequence between adjacent components
**5′ to 3′ orientation
†indicates a -1 frame-shift in the encoded construct (Gag-TFR-PR polyprotein)

The XDPs were designed to contain ribonucleoproteins (RNP) of CasX 676 complexed with single guide RNA variant 251 having spacer sequence 12.7 targeted to tdTomato (encoded by CTGCATTCTAGTTGTGGTTT, SEQ ID NO: 285) or spacer sequence 7.37 targeted to human beta 2 microglobulin (B2M). Utilizing methods described in the sections below, the XDPs were produced by transient transfection of LentiX HEK293T cells (Takara Biosciences) with two structural plasmids encoding components of the Gag-pol HIV-1 system, a plasmid encoding a pseudotyping glycoprotein, and a plasmid encoding the guide RNA. For the plasmid encoding the guide RNAs, the pStx42 plasmid was created with a human U6 promoter upstream of the guide RNA cassette A plasmid encoding a glycoprotein for pseudotyping the XDP was also used. All plasmids contained either an ampicillin or kanamycin resistance gene, were generated using standard molecular biology techniques, and were sequenced using Sanger sequencing to ensure correct assembly.

### Cell Culture and Transfection

HEK293T Lenti-X cells were maintained in 10% FBS supplemented DMEM with HEPES and Glutamax (Thermo Fisher). Cells were seeded in 15 cm dishes at 20×10⁶ cells per dish in 20 mL of media. Cells were allowed to settle and grow for 24 hours before transfection. At the time of transfection, cells were 70-90% confluent. For transfection, the XDP structural plasmids and the plasmid encoding CasX were used in amounts ranging from 13 to 80.0 μg. The structural plasmids and the plasmid encoding CasX were added at a ratio of 10:45:45 for plasmid 1:plasmid 2:plasmid 3. Each transfection also received 13 μg of the plasmid encoding the sgRNA and 0.25 μg of a plasmid encoding a glycoprotein. Polyethylenimine (PEI Max, Polyplus) was then added to the plasmid mixture, mixed, and allowed to incubate at room temperature before being added to the cell culture.

### Collection and Concentration of XDPs

Media was aspirated from the plates 24 hours post-transfection and replaced with Opti-MEM (Thermo Fisher). XDP-containing media was collected 72 hours post-transfection and filtered through a 0.45 μm PES filter. The supernatant was concentrated and purified via centrifugation at 10,000×g at 4° C. for 4h using a 10% sucrose buffer in NTE (50 mM Tris-HCL, 100 mM NaCl, 10% Sucrose, pH 7.4).

Pellets were either resuspended in Storage Buffer (PBS+113 mM NaCl, 15% Trehalose dihydrate, pH 8 or an appropriate media by gentle trituration and vortexing. XDPs were resuspended in 300 μL of DMEM/F12 supplemented with glutamax, HEPES, non-essential amino acids, Pen/Strep, 2-mercaptoethanol, B-27 without vitamin A, and N2.

### Transduction

For the experiments, XDPs were transduced into either mouse tdTomato neural progenitor cells, Jurkat T cells, human neural progenitor cells, or human astrocytes.

tdTomato neural progenitor cells (tdT NPCs) were grown in DMEM/F12 supplemented with glutamax, HEPES, non-essential amino acids, Pen/Strep, 2-mercaptoethanol, B-27 without vitamin A, and N2. Cells were harvested using StemPro Accutase Cell Dissociation Reagent and seeded on PLF coated 96-well plates. Cells were allowed to grow for 48 hours before being treated for targeting XDPs (having a spacer for tdTomato) starting with neat-resuspended virus and proceeding through 5 half-log dilutions. Cells were then centrifuged for 15 minutes at 1000×g. NPCs were grown for 96 hours before analysis of fluorescence as a marker of editing of tdTomato.

Human NPCs were grown in DMEM/F12 supplemented with glutamax, HEPES, non-essential amino acids, Pen/Strep, 2-mercaptoethanol, B-27 without vitamin A, and N2. Cells were harvested using StemPro Accutase Cell Dissociation Reagent and seeded on PLF coated 96-well plates. Cells were allowed to grow for 24 hours before being treated for targeting XDPs (having a spacer for tdTomato) starting with neat-resuspended virus and proceeding through 10 half-log dilutions. Cells were then centrifuged for 15 minutes at 1000×g. Human NPCs were grown for 96 hours before analysis of B2M editing by flow. The assays were run 2 times for each sample with similar results. Human astrocytes were similarly treated.

Jurkat cells were grown in RPMI supplemented with FBS. 20,000 cells were transduced with the targeting XDPs (having a spacer for tdTomato) starting with neat-resuspended virus and proceeding through 10 half-log dilutions. Cells were then centrifuged for 15 minutes at 1000×g. Jurakts were grown for 96 hours before analysis of B2M editing by flow. The assays were run 2 times for each sample with similar results.

tdTomato fluorescence and editing of the B2M locus was measured using flow cytometry. To measure B2M editing, 4′,6-diamidino-2-phenylindole (DAPI) staining was used to mark dead cells, and the PE-Cy7 Mouse Anti-Human HLA-ABC staining kit (BD Pharmingen) was used to stain major the histocompatibility complex, class I. Expression of this complex at the cell surface is blocked by B2M knockout. The assays were run 2-3 times for each sample, with similar results.

### Results

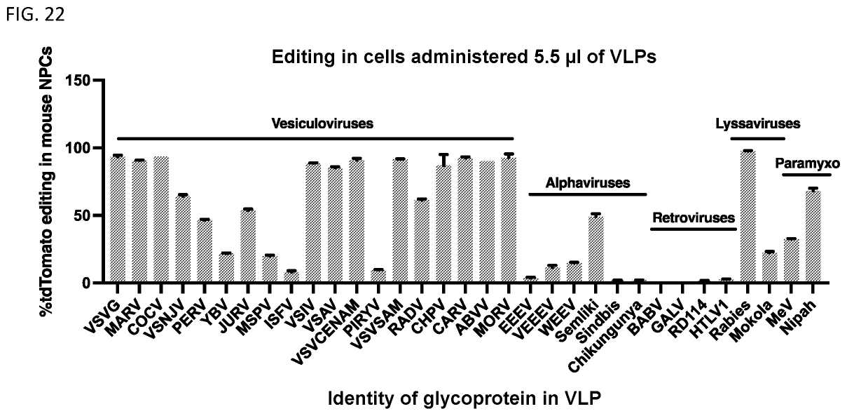

VSV-G-mediated cell entry occurs by binding to the low-density lipoprotein receptor (LDL-R), which is a ubiquitous receptor found on most cell types. Accordingly, the tropism of XDPs pseudotyped with VSV-G is broad. In order to alter the tropism of XDPs relative to XDPs pseudotyped with VSV-G, XDPs were generated with diverse viral glycoproteins as targeting moieties. These XDPs were transduced into mouse tdTomato neural progenitor cells (NPCs), Jurkat T cells, human NPCs, or human astrocytes, and editing of the tdTomato or human B2M locus was measured to determine differences in tropism conferred by the different glycoproteins (FIGS. 22-25). For example, it was expected that the glycoproteins from neurotropic viruses, such as alphaviruses and lyssaviruses, would display tropism for neuronal cells over other cell types, such as Jurkat cells.

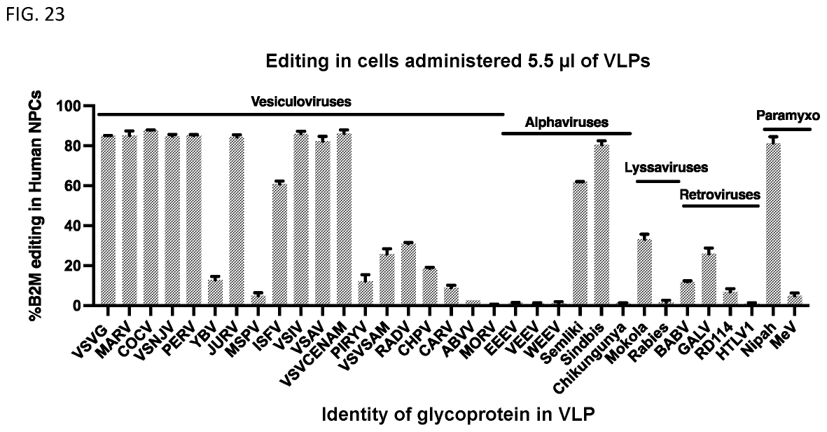

A comparison of the mouse and human NPC editing data revealed that the XDPs did not edit mouse and human NPCs at the same levels. Specifically, almost all of the XDPs with vesiculoviral glycoproteins showed a higher level of editing in mouse NPCs (FIG. 22) than they did in human NPCs (FIG. 23). XDPs with alphaviral glycoproteins showed a higher level of editing in human NPCs than in mouse NPCs. Interestingly, XDPs with rabies glycoprotein showed a higher level of editing in mouse NPCs than in human NPCs. Conversely, XDPs with Mokola glycoprotein showed a higher level of editing in human NPCs than in mouse NPCs.

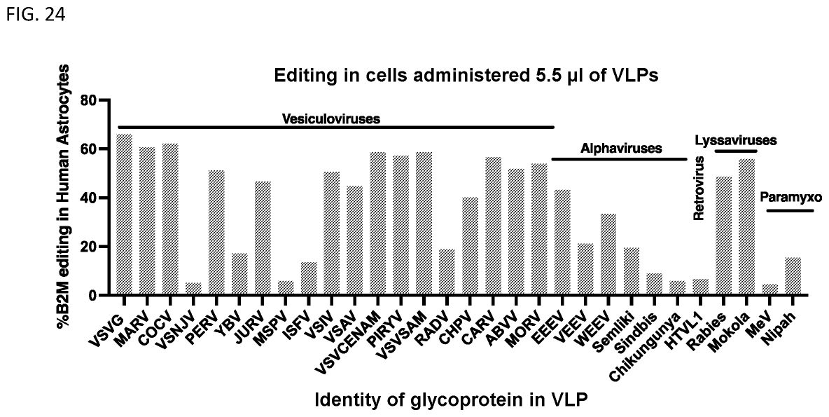

Additionally, XDPs with certain glycoproteins belonging to the vesiculoviral family (including PERV, YBV, JURV, PIRYV, RADV and CHIPV) showed higher levels of editing in human astrocytes (FIG. 24) than in human NPCs (FIG. 23). This finding may be particularly useful to skew XDP tropism towards glial cells rather than neurons, which would be beneficial for glial cell targets.

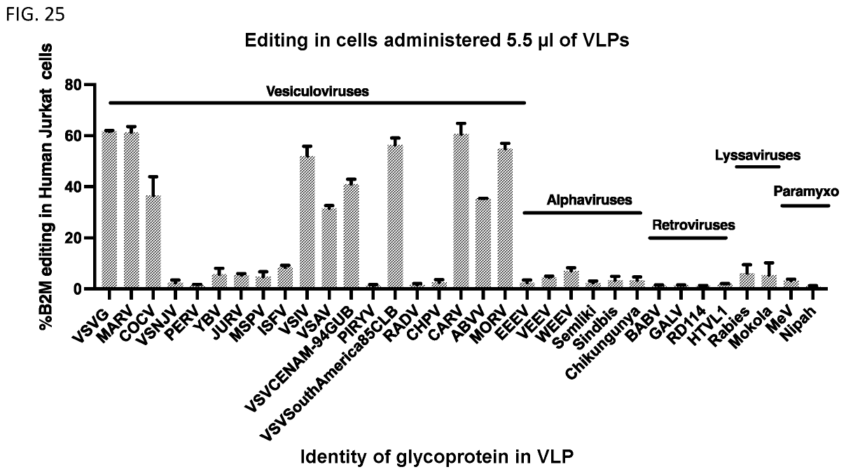

Finally, the level of editing of the B2M locus was measured in Jurkat cells, a human T lymphocyte cell line. Only XDPs with certain glycoproteins belonging to the vesiculoviral family showed a higher level of editing in Jurkat cells (FIG. 25), with minimal editing in Jurkat cells exhibited by XDPs with glycoproteins belonging to lyssaviruses, alphaviruses, paramyxoviruses and retroviruses.

The results of the experiments support that viral glycoproteins can be selectively utilized in glycoprotein particles to preferentially confer tropism on cells intended for gene editing.

What is claimed is:

1. A glycoprotein particle comprising a particle and a glycoprotein, wherein the glycoprotein is any one of the sequences of SEQ ID NOS: 1-224, 281-284, or 286-573 as set forth in Table 1, a sequence comprising at least 70%, at least 80%, at least 90%, at least 95%, at least 96%, at least 97%, at least 98%, or at least 99% identity thereto, or a fragment thereof.

2. The glycoprotein particle of claim 1, wherein the glycoprotein consists of any one of SEQ ID NOS: 1-224, 281-284, or 286-573 as set forth in Table 1.

3. The glycoprotein particle of claim 1, wherein the glycoprotein fragment is a virion surface domain.

4. The glycoprotein particle of claim 1, wherein the particle is a microparticle.

5. The glycoprotein particle of claim 1, wherein the particle is a nanoparticle.

6. The glycoprotein particle of claim 1, wherein the particle comprises one or more of fullerenes, metals, biological polymers, dendrimers, quantum dots, lipids, nucleic acid vectors, and viral structural proteins.

7. The glycoprotein particle of claim 5, wherein the particle is a polymeric nanoparticle.

8. The glycoprotein particle of claim 7, wherein the polymeric nanoparticle comprises a nanocapsule or a nanosphere.

9. The glycoprotein particle of claim 7, wherein the polymeric nanoparticle comprises a polymer micelle.

10. The glycoprotein particle of any one of claims 1-9, wherein the particle is non-toxic and/or biodegradable.

11. The glycoprotein particle of any one of claims 1-5, wherein the particle is a lipid-based particle.

12. The glycoprotein particle of claims 1-3, wherein the particle is a virus-like particle (VLP).

13. The glycoprotein particle of claim 12, wherein the VLP is derived from a virus selected from the group consisting of retrovirus, lentivirus, adeno-associated virus (AAV), adenovirus, and herpes simplex virus.

14. The glycoprotein particle of claim 13, wherein the retrovirus is an Orthoretrovirinae virus or a Spumaretrovirinae virus.

15. The glycoprotein particle of claim 14, wherein the Orthoretrovirinae virus is selected from the group consisting of an Alpharetrovirus, Betaretrovirus, Deltaretrovirus, Epsilonretrovirus, Gammaretrovirus, and Lentivirus.

16. The glycoprotein particle of claim 14, wherein the Spumaretrovirinae virus is selected from the group consisting of Bovispumavirus, Equispumavirus, Felispumavirus, Prosimiispumavirus, Simiispumavirus, or Spumavirus.

17. The glycoprotein particle of claims 1-3, wherein the particle is an exosome.

18. The glycoprotein particle of any one of claim 1-3, or 12-17, wherein the glycoprotein is expressed in a host cell and is trafficked to the host cell membrane prior to formation of the glycoprotein particle.

19. The glycoprotein particle of claims 1-3, wherein the particle comprises a lipid nanoparticle (LNP).

20. The glycoprotein particle of any one of claims 1-19, wherein the glycoprotein is incorporated into a membrane of the particle.

21. The glycoprotein particle of any one of claims 1-20, wherein the glycoprotein particle comprises an organic polymer-based particle.

22. The glycoprotein particle of any one of claims 1-21, wherein the particle comprises an inorganic nanoparticle.

23. The glycoprotein particle of any one of claim 1-22, wherein the glycoprotein is conjugated to the surface of the particle.

24. The glycoprotein particle of claim 23, wherein the conjugation comprises covalent attachment or non-covalent attachment.

25. The glycoprotein particle of claims 1-24, wherein the glycoprotein particle comprises a payload.

26. The glycoprotein particle of claim 25, wherein inclusion of the glycoprotein in the glycoprotein particles enhances delivery of the payload to a cell or tissue compared to an equivalent particle that does not comprise the glycoprotein.

27. The glycoprotein particle of claims 24-26, wherein the payload comprises a protein, a nucleic acid, a small molecule, or a combination thereof.

28. The glycoprotein particle of claims 25-27, wherein the payload is a therapeutic payload.

29. The glycoprotein particle of claim 28, wherein delivery of the therapeutic payload to a cell or a tissue of a subject treats a disease or disorder in the subject.

30. The glycoprotein particle of claim 28 or 29, wherein the therapeutic payload comprises a protein or an oligonucleotide.

31. The glycoprotein particle of claim 30, wherein the oligonucleotide comprises a sequence encoding a protein.

32. The glycoprotein particle of claim 30 or 31, wherein the protein comprises a cytokine, growth factor, interleukin, enzyme, receptor, microprotein, hormone, RNAse, DNAse, blood clotting factor, anticoagulant, bone morphogenetic protein, engineered protein scaffold, thrombolytics, antibody, antibody fragment, antibody fusion protein, transcription factor, viral interferon antagonist, tick protein, or engineered therapeutic protein.

33. The glycoprotein particle of claim 30, wherein the oligonucleotide comprises a single-stranded antisense oligonucleotide (ASO), double-stranded RNA interference (RNAi) molecule, DNA aptamer, RNA aptamer, microRNA, ribozyme, RNA decoy, or circular RNA.

34. The glycoprotein particle of claims 25-27, wherein the payload is a diagnostic payload.

35. The glycoprotein particle of any one of claims 25-27, wherein the payload comprises a gene editing system.

36. The glycoprotein particle of claim 35, wherein the gene editing system comprises a Class 2, Type II CRISPR/Cas system, a Class 2, Type V CRISPR/Cas system, a zinc finger nuclease or a TALEN.

37. The glycoprotein particle of claim 36, wherein the Class 2, Type II CRISPR/Cas system comprises Cas9.

38. The glycoprotein particle of claim 36, wherein the Class 2, Type V CRISPR/Cas system comprises one or more of CasX, Cas12a (Cpf1), Cas12b (C2c1), Cas12c (C2c3), Cas12d (CasY), Cas12e (CasX), Cas12f, Cas12g, Cas12h, Cas12i, Cas12j, Cas12k, Cas12l, Cas14, and/or CasΦ.

39. The glycoprotein particle of claim 38, wherein the Class 2, Type V CRISPR/Cas system comprises a gene editing pair comprising a CasX protein comprising an amino acid sequence with at least 90% identity to any one of SEQ ID NOS: 574-944, or as set forth in Table 2, and a guide RNA (gRNA) comprising a scaffold comprising a nucleic acid sequence with at least 90% identity to any one of SEQ ID NOS: 945-1141, or as set forth in Table 3.

40. The glycoprotein particle of any one of claims 25-30, wherein the payload comprises a gene editing pair comprising a CasX protein of any one of SEQ ID NOS: 574-944, or as set forth in Table 2, and a guide RNA comprising a scaffold of any one of SEQ ID NOS: 945-1141, or as set forth in Table 3.

41. The glycoprotein particle of claim 39 or 40, wherein the gRNA further comprises a targeting sequence linked to the 3′ end of the gRNA scaffold sequence that is complementary to a target nucleic acid.

42. A pharmaceutical composition comprising a glycoprotein particle of any one of the preceding claims and a pharmaceutically acceptable carrier, diluent, or excipient.

43. A method of treating a subject in need thereof, comprising administering to a subject in need thereof, a glycoprotein particle of any one of claims 1-41 or the pharmaceutical composition of claim 42.

44. The method of claim 43, wherein the subject has a disease or disorder selected from the group consisting of cancer, an immunoregulatory disease, a pulmonary disease or disorder, a cardiovascular disease, an infectious disease, a genetic disease or disorder, a neurological disease or disorder, an endocrine disease or disorder, a metabolic disease or disorder, an intestinal disease or disorder, a mental illness, a sexually transmitted disease, a gynecological disease, an urogenital disease, a skin disease, or an ocular disease.

45. The method of claim 43 or 44, wherein administration of the glycoprotein particle or pharmaceutical composition reduces a sign or a symptom of the disease or disorder.
